(19) United States
(12) Patent Application Publication
(10) Pub. No.: US 2004/0247672 A1
(43) Pub. Date: Dec. 9, 2004

(54) Injectable sustained release compositions

(75) Inventors: Mark A. Tracy, Arlington, MA (US); Chiem V. Pham, Mason, OH (US); Firouz Asgarzadeh, Hillsborough, NJ (US); J. Don Wang, Frontenac, MO (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Alkermes Controlled Therapeutics, Inc., Cambridge, MA

(21) Appl. No.: 10/843,872

(22) Filed: May 12, 2004

Related U.S. Application Data
(60) Provisional application No. 60/471,209, filed on May 16, 2003.

(51) Int. Cl. A61K 9/22

(52) U.S. Cl. 424/468

(57) ABSTRACT

The present invention relates to a polymer paste and a sustained release composition comprising the paste and a biologically active agent. The polymer paste comprises a biocompatible, biodegradable polymer having an inherent viscosity of about 0.12 dL/g or less and a viscosity reducing agent, wherein the biocompatible, biodegradable polymer is present in the polymer paste in at least 60% by weight and the viscosity of the paste is about 400 cP or less. The sustained release composition comprises a biologically active agent and a polymer paste comprising a biocompatible, biodegradable polymer having an inherent viscosity of about 0.12 dL/g or less and a viscosity reducing agent, wherein the biocompatible, biodegradable polymer is present in the polymer paste in at least 60% by weight and the viscosity of the sustained release composition is about 400 cP or less. In a particular embodiment, the sustained release composition is injectable.

### RELATED APPLICATION

This application claims the benefit of U.S. Provisional Patent Application No. 60/471,209, filed May 16, 2003, the entire teachings of which are incorporated herein by reference.

### BACKGROUND OF THE INVENTION

Many illnesses or conditions require administration of a constant or sustained level of a medicament or biologically active agent to provide the desired prophylactic or therapeutic effect. This release can be accomplished through a multiple dosing regimen or by employing a system that releases the medicament in a sustained fashion.

Attempts to sustain medication levels include the use of biodegradable materials, such as polymeric matrices, containing the medicament. The use of these matrices, for example, in the form of microparticles or microcarriers, provides sustained release of medicaments by utilizing the inherent biodegradability of the polymer. The ability to provide a sustained level of medicament can result in improved patient compliance. For example, patient compliance can be particularly difficult in the treatment of chronic disorders or diseases.

Certain methods of fabricating injectable polymer-based sustained release devices comprise the steps of dissolving a polymer in a solvent, adding the active agent to be incorporated to the polymer solution and removing the solvent from the mixture, thereby forming a matrix of the polymer in a size suitable for injection with the active agent distributed throughout. However, the physical characteristics of the injectable composition (microparticles), such as the morphology, density and size, are significantly dependent upon all steps used in the method of preparation, making control and tailoring of the physical characteristics of the resulting microparticles a difficult and expensive undertaking.

Other injectable compositions for providing a sustained release of biologically active agent include polymer/drug mixtures which are delivered to the body in a fluid state. Once in the body, the compositions coagulate or cure to form a solid implant. However, these compositions can require the use of large amounts of organic solvents to permit flowability. The use of such large amounts of organic solvents raises safety concerns, particularly for treatment of chronic conditions. In addition, the formation of the solid matrix in vivo from the flowable system is generally not instantaneous causing the biologically active agent to diffuse from the coagulating polymer resulting in undesirable release.

In view of the above, there is a need for improved, injectable polymer-based sustained release compositions.

### SUMMARY OF THE INVENTION

The present invention relates to a polymer paste and a sustained release composition comprising the paste and a biologically active agent. The polymer paste comprises a biocompatible, biodegradable polymer having an inherent viscosity of about 0.12 deciliters/gram (dL/g) or less and a viscosity reducing agent, wherein the biocompatible, biodegradable polymer is present in the polymer paste in at least 60% by weight and the viscosity of the paste is about 400 centipoise (cP) or less. The sustained release composition comprises a biologically active agent and a polymer paste comprising a biocompatible, biodegradable polymer having an inherent viscosity of about 0.12 dL/g or less and a viscosity reducing agent, wherein the biocompatible, biodegradable polymer is present in the polymer paste in at least 60% by weight and the viscosity of the sustained release composition is about 400 cP or less. In a particular embodiment, the sustained release composition is injectable.

The invention further relates to a method of delivering an active agent in a sustained fashion to a patient in need thereof comprising administering a therapeutically effective amount of a sustained release composition comprising a biologically active agent and a polymer paste comprising a biocompatible, biodegradable polymer having an inherent viscosity of about 0.12 dL/g or less and a viscosity reducing agent, wherein the biocompatible, biodegradable polymer is present in the polymer paste in at least 60% by weight and the viscosity of the composition is about 400 cP or less. In a particular embodiment, the sustained release composition is administered by injection.

In one embodiment, the viscosity reducing agent can be selected from the group consisting of polyethylene glycol polymers, polymer surfactants (hydrophilic, hydrophobic or amphiphilic), organic solvents, aqueous solvents, and combinations thereof.

In another embodiment, the base polymer is a poly(lactide-co-glycolide) polymer. For example, the poly(lactide-co-glycolide) polymer can be TEGPLGA5050 (Tetraethylene glycol poly(lactide-co-glycolide)): 50%/50% lactide/glycolide monomer units in copolymer and tetraethylene glycol as initiator; ULMPLGA5050-L (Ultralow molecular weight poly(lactide-co-glycolide)): 50%/50% lactide/glycolide monomer units in copolymer and lauryl alcohol as initiator; ULMPLGA5050-LA (Ultralow molecular weight poly(lactide-co-glycolide)): 50%/50% lactide/glycolide monomer units in copolymer and lauric acid as the initiator; ULMPLGA7525-L (Ultralow molecular weight poly(lactide-co-glycolide)): 75%/25% lactide/glycolide monomer units in copolymer and lauryl alcohol as the initiator; or ULMPDLLA-L (Ultralow molecular weight poly((D,L-)lactide)): 100% lactide monomer units in polymer and lauryl alcohol as the initiator.

The use of biocompatible, biodegradable polymers having an inherent viscosity of about 0.12 dL/g or less results in the ability to use a greater weight percent of the polymer (e.g., about 60 wt % or more) in the paste of the sustained release composition while maintaining suitable flowability for injection, since less of the viscosity reducing agent, e.g., about 40 wt % or less, is needed in the compositions of the present invention to achieve a viscosity which renders the compositions suitable for injection (e.g., 400 cP or less). As a result, the use of a greater weight percent of polymer can provide sustained release compositions with an increased drug load.

A suitable viscosity is about 400 cP or less, for example, about 300 cP or less. In a particular embodiment, the viscosity is about 200 cP or less, for example, about 100 cP or less such as about 50 cP or less. The value of viscosity as used herein is determined at 37° C.

The injectable polymer-based sustained release composition of the present invention provides a facile manufacturing process by eliminating the need to form compositions with a defined shape, for example, microparticles for injection. Advantageously, the system permits a higher drug load thereby reducing the injection volume and frequency. In addition, the base polymers described herein can provide a sustained release composition with suitable flowability and injectability resulting in a desired suspension of drugs in the polymer, thereby decreasing the surface area and subsequently decreasing the surface drug concentration which can result in reduced initial drug release. A further advantage is that the base polymers described herein permit a higher concentration of polymer, resulting in improved sustained release of the active agent.

### BRIEF DESCRIPTION OF THE DRAWINGS

The foregoing and other objects, features and advantages of the invention will be apparent from the following more particular description of preferred embodiments of the invention, as illustrated in the accompanying drawings.

FIG. 1 is a graph of weight loss percent versus time in days for the indicated base polymer and polymer pastes.

FIG. 2 is a bar graph showing the viscosities of polymer pastes prepared using TEGPLGA5050 at 60% and the indicated viscosity reducing agent at 40%.

FIG. 3 is a bar graph showing the viscosities of polymer pastes prepared using 40% PEG200 as the viscosity reducing agent and the indicated base polymer at 60%.

FIG. 4 is a bar graph showing the viscosities of polymer pastes (P1-P9) prepared using the indicated base polymer at 60% and the indicated viscosity reducing agent at 40%.

FIG. 5 is a bar graph showing the viscosities of polymer pastes having the indicated weight percent of ULMPLGA5050-2L as the base polymer with the remaining weight percent being acetic acid as the viscosity reducing agent.

FIG. 6 is a bar graph showing the viscosities of polymer pastes (P10-P21) prepared from the indicated combinations of base polymer (60 wt %) and viscosity reducing agent (40 wt %).

FIG. 7 is a bar graph showing the viscosities of polymer pastes (P24-P26) prepared from the indicated combinations of base polymer and viscosity reducing agent.

FIG. 8 is a graph of viscosity versus temperature for the indicated polymer pastes.

FIG. 9 is a graph of viscosity versus shear stress for the indicated polymer pastes.

FIG. 10 is a graph of percent cumulative release of insulin versus time for sustained release compositions I3 and I13 comprising insulin, the indicated polymer at 60 wt % and PEG200 at 40 wt %.

FIG. 11 is a graph of % cumulative release of insulin versus time for sustained release compositions I11 and I16 comprising insulin, the indicated polymer at 60 wt % and a combination of 20 wt % DMSO+20 wt % acetic acid as the viscosity reducing agent.

FIG. 12 is a graph of percent cumulative release of insulin versus time for sustained release compositions I23 and I24 comprising insulin, the indicated polymers at 60 wt % and a combination of 20 wt % DMSO+20% acetic acid as the viscosity reducing agent.

FIG. 13 is a graph of percent cumulative release of insulin versus time for sustained release compositions I3, I6 and I8 comprising insulin, ULMPLGA5050-2L as the base polymer at 60 wt %.

FIG. 14 is a graph of percent cumulative release of insulin versus time for sustained release compositions I9, I10, I11 and I12 comprising insulin, ULMPLGA5050-2L as the base polymer at 60 wt %.

FIG. 15 is a graph of percent cumulative release of insulin versus time for sustained release compositions I14, I15, I16 and I17 comprising insulin, ULMPDLLA as the base polymer at 60 wt %.

FIG. 16 is a graph of percent cumulative release of insulin versus time for sustained release compositions I1, I2 and I3 comprising insulin, ULMPLGA5050-2L as the base polymer and PEG200 as the viscosity reducing agent.

FIG. 17 is a graph of the percent cumulative release of insulin versus time for sustained release compositions I1, I5 and I6 comprising insulin, ULMPLGA5050-2L as the base polymer and DMSO as the viscosity reducing agent.

FIG. 18 is a graph of the percent cumulative release of insulin versus time for sustained release compositions I1, I7 and I8 comprising insulin, ULMPLGA5050-2L as the base polymer and acetic acid as the viscosity reducing agent.

FIG. 19 is a graph of the percent cumulative release of naltrexone versus time for naltrexone alone (50 micrograms, No paste) and a sustained release composition N2 comprising naltrexone (20 g/100 g paste), ULMPLGA-2L as the base polymer and PEG200 as the viscosity reducing agent.

FIG. 20 is a graph of the percent cumulative release of naltrexone versus time for sustained release compositions comprising naltrexone, 60 wt % ULMPLGA5050-2L+40 wt % PEG200 as the polymer paste and having a drug load of 20 g/100 g of polymer paste (N2) and 50 g/100 g of polymer paste (N1).

FIG. 21 is a graph of the percent cumulative release of naltrexone versus time for sustained release compositions N11, N15, N19 and N20, comprising naltrexone and a mixture of 20 wt % DMSO+20 wt % acetic acid as the viscosity reducing agent.

FIG. 22 is a graph of the percent cumulative release of naltrexone versus time for sustained release compositions N2, N12 and N16, comprising naltrexone, 40 wt % PEG200 as the viscosity reducing agent.

FIG. 23 is a graph of the percent cumulative release of naltrexone versus time for sustained release compositions N2, N3 and N4, comprising naltrexone, PEG200 as the viscosity reducing agent and ULMPLGA5050-2L as the base polymer.

FIG. 24 is a graph of the percent cumulative release of naltrexone versus time for sustained release compositions N3, N6 and N7, comprising naltrexone, DMSO as the viscosity reducing agent and ULMPLGA5050-2L as the base polymer.

FIG. 25 is a graph of the percent cumulative release of naltrexone versus time for sustained release compositions N3 and N8 comprising naltrexone, acetic acid as the viscosity reducing agent and ULMPLGA5050-2L as the base polymer.

FIG. 26 is a graph of the percent cumulative release of naltrexone versus time for sustained release compositions N19, N20 and N21, comprising naltrexone and a combination of 20 wt % DMSO+20 wt % acetic acid as the viscosity reducing agent.

FIG. 27 is a graph of the percent cumulative release of naltrexone versus time for sustained release compositions N2, N7 and N8, comprising naltrexone, ULMPLGA5050-2L at 60 wt %.

FIG. 28 is a graph of the percent cumulative release of naltrexone versus time for sustained release compositions N9, N10 and N11, comprising naltrexone, ULMPLGA5050-2L at 60 wt %.

FIG. 29 is a graph of the percent cumulative release of naltrexone versus time for sustained release compositions, N13, N14 and N15, comprising naltrexone, ULMPDLLA at 60 wt %.

FIG. 30 is a graph of the percent cumulative release of naltrexone versus time for sustained release compositions N17, N18 and N19, comprising naltrexone, TEGPLGA at 60 wt %.

### DETAILED DESCRIPTION OF THE INVENTION

A description of preferred embodiments of the invention follows.

The present invention relates to a polymer paste and a sustained release composition comprising the paste and a biologically active agent. The polymer paste comprises a biocompatible, biodegradable polymer having an inherent viscosity of about 0.12 dL/g or less and a viscosity reducing agent, wherein the biocompatible, biodegradable polymer is present in the polymer paste in at least 60% by weight and the viscosity of the paste is about 400 cP or less. The sustained release composition comprises a biologically active agent and a polymer paste comprising a biocompatible, biodegradable polymer having an inherent viscosity of about 0.12 dL/g or less and a viscosity reducing agent, wherein the biocompatible, biodegradable polymer is present in the polymer paste in at least 60% by weight and the viscosity of the sustained release composition is about 400 cP or less. In a particular embodiment, the sustained release composition is injectable. The viscosity of the sustained release composition can be about 400 cP or less, thereby providing flowability characteristics to permit injection. For example, a viscosity of about 300 cP or less, about 200 cP or less, about 100 cP or less or about 50 cP or less can provide a sustained release composition suitable for injection. The biodegradable, biocompatible polymer of the polymer paste is referred to herein as the base polymer.

In one embodiment, the viscosity reducing agent can be selected from the group consisting of polyethylene glycol polymers, surfactants (hydrophilic, hydrophobic or amphiphilic), organic solvents, aqueous solvents, and combinations thereof.

In another embodiment, the base polymer is a poly(lactide-co-glycolide) polymer. For example, the poly(lactide-co-glycolide) polymer can be TEGPLGA5050 (Tetraethylene glycol poly(lactide-co-glycolide)): 50%/50% lactide/glycolide monomer units in copolymer and tetraethylene glycol as initiator; ULMPLGA5050-L and ULMPLGA5050-L (Ultralow molecular weight poly(lactide-co-glycolide)): 50%/50% lactide/glycolide monomer units in copolymer and lauryl alcohol as initiator; ULMPLGA5050-LA (Ultralow molecular weight poly(lactide-co-glycolide)): 50%/50% lactide/glycolide monomer units in copolymer and lauric acid as the initiator; ULMPLGA7525-L (Ultralow molecular weight poly(lactide-co-glycolide)): 75%/25% of lactide/glycolide monomer units in copolymer and lauryl alcohol as the initiator; or ULMPDLLA-L (Ultralow molecular weight poly((D,L-)lactide)): 100% of lactide monomer units in polymer and lauryl alcohol as the initiator.

Viscosity Reducing Agents

Suitable viscosity-reducing agents for use in the present invention are bioresorbable, biocompatible and miscible with the polymer of the resulting polymer paste. It is desirable that the viscosity reducing agents possess a low viscosity, for example, a viscosity of about 400 cP or less. It is preferred that the solubility characteristics of the viscosity reducing agent are similar to those of the base polymer. Combinations of viscosity reducing agents are suitable for use in the invention.

For example, polyethylene glycol polymers, surfactants, organic solvents, aqueous solvents and combinations thereof are suitable for use as viscosity reducing agents. The amount of viscosity reducing agent present in the sustained release composition of the invention can range from about 5 wt % to about 40 wt % based on the combined weight of the base polymer and viscosity reducing agent.

Polyethylene glycol polymers (PEG) are liquid and solid polymers of the general formula H(OCH₂CH₂)ₙOH, where n is greater than or equal to 4. In general, each PEG is followed by a number which corresponds to its average molecular weight. For example, PEG200 has an average value of n of 4 with a molecular weight range between 190 and 210, PEG400 has an average value of n between 8.2 and 9.1 with a molecular weight range between 380-420, PEG600 has an average value of n between 12.5 and 13.9 with a molecular weight range between 570 and 630. In a particular embodiment, the PEG is a liquid at room temperature. For example, the PEG can be PEG200, PEG400 or MPEG350 (monomethoxy PEG) which are all liquids at room temperature.

Polymer surfactants, in particular, nonionic polymer surfactants, are suitable for use in the invention. Suitable nonionic polymer surfactants include poloxamers, which are polyethylenepolypropyleneglycol polymers commonly referred to as Pluronics. Suitable examples include, but are not limited to, poloxamer 331, poloxamer 407 sold under the trademark PLURONIC® F127, poloxamer 188 sold under the trademark PLURONIC® F68, poloxamer 184 sold under the trademark PLURONIC® L64, PLURONIC® L31, PLURONIC® L101, and combinations thereof. PLURONIC is a trademark of BASF Corp. (Mount Olive, N.J.).

Polysorbates are another type of nonionic surfactant often referred to as polyoxyethylene sorbitan esters. Polysorbate 80 sold under the trademark TWEEN® 80, polysorbate 20 sold under the trademark TWEEN® 20, and combinations thereof are suitable polysorbates for use in the invention. TWEEN® is a trademark of ICI Americas, Inc. (Bridgewater, N.J.).

Suitable organic solvents for use as a viscosity reducing agent are biocompatible, pharmaceutically acceptable and miscible to dispersible in aqueous or body fluids. In addition, the pharmaceutically acceptable organic solvents are present in the sustained release composition in an amount which is pharmaceutically acceptable. Organic solvents suitable for use as a viscosity reducing agent include, but are not limited to, organic acids such as lactic acid and acetic acid; dimethyl sulfoxide (DMSO); dimethylsulfone; tetrahydrofuran; N-methyl-2-pyrrolidone (NMP); 2-pyrrolidone; alcohols such as solketal, glycerol formal, benzyl alcohol and glycofurol; dialkylamides such as dimethylformamide, dimethylacetamide; triacetiens; and other suitable solvents such as benzyl benzoate, methyl benzoate, ethyl acetate, ethyl lactate and any combinations thereof.

Combinations of viscosity reducing agents which are suitable for use in the invention include, but are not limited to, PEG and an organic solvent, PEG and an organic acid, and/or two or more organic solvents, for example, DMSO and acetic acid.

Base Polymers

Base polymers suitable to form the polymer paste and the sustained release composition of this invention are biocompatible, biodegradable polymers, blends or copolymers thereof having an inherent viscosity of about 0.12 dL/g or less. A polymer is biocompatible if the polymer and any degradation products of the polymer are non-toxic to the recipient and also possess no significant deleterious or untoward effects on the recipient's body, such as an immunological reaction at the injection site. The use of a polymer having an inherent viscosity of about 0.12 dL/g or less permits the polymer to be present in the paste in an amount suitable to impart sustained release to the incorporated active agent of the sustained release composition, while providing a suitable viscosity for administration of the composition by injection. It has been determined that polymers having an inherent viscosity of about 0.12 dL/g or less and which are present in the paste in at least 60 wt % can impart sustained release characteristics to the composition.

Biodegradable, as defined herein, means the composition will degrade or erode in vivo to form smaller units or chemical species. Degradation can result, for example, by enzymatic, chemical and physical processes.

The terminal functionalities or pendant groups of the polymers can be modified, for example, to modify hydrophobicity, hydrophilicity and/or to provide, remove or block moieties which can interact with the active agent via, for example, ionic or hydrogen bonding.

Suitable biocompatible, biodegradable polymers include, for example, poly(lactide)s, poly(glycolide)s, poly(lactide-co-glycolide)s, poly(lactic acid)s, poly(glycolic acid)s, polycarbonates, polyesteramides, polyanhydrides, poly(amino acid)s, polyacetals, polycyanoacrylates, polyetheresters, polyorthoesters, polycaprolactone, poly(dioxanone)s, poly(alkylene alkylate)s, polyurethane and blends and copolymers thereof, for example, PLG-co-EMPO polymers described in copending U.S. patent application Ser. No. 09/886,394, entitled “Functionalized Degradable Polymer,” filed on Jun. 22, 2001, the entire content of which is hereby incorporated by reference.

Acceptable base polymers used in this invention can be determined by a person of ordinary skill in the art taking into consideration factors such as the viscosity of the resulting sustained release composition, the desired polymer degradation rate, physical properties such as mechanical strength, end group chemistry and rate of dissolution of polymer in solvent. In a preferred embodiment, the base polymer is a poly(lactide-co-glycolide) (hereinafter “PLG” or “PLGA”). In particular embodiments, the poly(lactide-co-glycolide) contains free carboxyl end groups. In other embodiments, the poly(lactide-co-glycolicde) contains alkyl ester end groups such as methyl ester end groups and lauryl ester end groups.

Specific poly(lactide-co-glycolide) polymers suitable for use in the invention include, but are not limited to: TEGPLGA5050 (Tetraethylene glycol poly(lactide-co-glycolide)): 50%/50% lactide/glycolide monomer units in copolymer and tetraethylene glycol as initiator; ULMPLGA5050-1L and ULMPLGA5050-2L (Ultralow molecular weight poly(lactide-co-glycolide)): 50%/50% lactide/glycolide monomer units in copolymer and lauryl alcohol as initiator; ULMPLGA5050-1LA (Ultralow molecular weight poly(lactide-co-glycolide)): 50%/50% lactide/glycolide monomer units in copolymer and lauric acid as the initiator; ULMPLGA7525-1L (Ultralow molecular weight poly(lactide-co-glycolide)): 75%/25% of lactide/glycolide monomer units in copolymer and lauryl alcohol as the initiator; and ULMPDLLA-1L (Ultralow molecular weight poly((D,L-)lactide)): 100% of lactide monomer units in polymer and lauryl alcohol as the initiator.

The inherent viscosity, as used herein is the natural logarithm of the relative viscosity divided by the concentration of the polymer solution. The inherent viscosity is related to polymer molecular size as longer polymer chains result in larger values of inherent viscosity for a given polymer and solvent system.

The relative viscosity is the ratio of the viscosity of a polymer solution to the viscosity of the pure solvent. The relative viscosity is measured as the ratio of their efflux times (time required for a liquid to pass between the upper and lower graduation marks on a viscometer) through a standard capillary.

The inherent viscosity can be determined using the following calculation:

Inherent Viscosity (dL/g)=ln(RV)*1000*1/w=ln(RV)*1000/w

ln(RV)=natural logarithm of the relative viscosity

w=weight of polymer in the sample solution (mg)

The polymer paste and sustained release composition comprising the polymer paste and biologically active agent has an inherent viscosity of about 0.12 dL/g or less. For example, the inherent viscosity can be 0.12 or less, such as about 0.1 dL/g or less, for example, 0.1, 0.09, 0.08, 0.07, 0.06, 0.05, 0.04, 0.03, 0.02 and 0.01 dL/g when measured in chloroform at 30° C.

Further details relating to determination of the inherent viscosity are set forth in the Experimental Section.

Biologically Active Agents

The term “biologically active agent,” as used herein, is an agent or its pharmaceutically acceptable salt which, when released in vivo, possesses the desired biological activity, for example, therapeutic, diagnostic and/or prophylactic properties in vivo. It is understood that the term includes stabilized biologically active agents as described herein. The terms “biologically active agent,” “therapeutic, prophylactic or diagnostic agent,” “drug,” “active agent,” and “agent” are used interchangeably herein.

Examples of suitable biologically active agents include, but are not limited to, antipsychotic agents such as aripiprazole, risperidone, and olanzapine; antitumor agents such as bleomycin hydrochloride, carboplatin, methotrexate and adriamycin; antibiotics such as gentamicin, tetracycline hydrochloride and ampicillin; antipyretic, analgesic and anti-inflammatory agents; antitussives and expectorants such as ephedrine hydrochloride, methylephedrine hydrochloride, noscapine hydrochloride and codeine phosphate; sedatives such as chlorpromazine hydrochloride, prochlorperazine hydrochloride and atropine sulfate; muscle relaxants such as tubocurarine chloride; antiepileptics such as sodium phenytoin and ethosuximide; antiulcer agents such as metoclopramide; antidepressants such as clomipramine; antiallergic agents such as diphenhydramine; cardiotonics such as theophillol; antiarrhythmic agents such as propranolol hydrochloride; vasodilators such as diltiazem hydrochloride and bamethan sulfate; hypotensive diuretics such as pentolinium and ecarazine hydrochloride; antidiuretic agents such as metformin; anticoagulants such as sodium citrate and sodium heparin; hemostatic agents such as thrombin, menadione sodium bisulfite and acetomenaphthone; antituberculous agents such as isoniazide and ethanbutol; hormones such as prednisolone sodium phosphate and methimazole; and narcotic antagonists such as nalorphine hydrochloride.

Additional biologically active agents suitable for use in the invention include, but are not limited to, proteins, muteins and active fragments thereof, such as immunoglobulins, antibodies, cytokines (e.g., lymphokines, monokines, chemokines), interleukins, interferons (β-IFN, α-IFN and γ-IFN), erythropoietin, nucleases, tumor necrosis factor, colony stimulating factors, insulin, enzymes (e.g., superoxide dismutase, tissue plasminogen activator), tumor suppressors, blood proteins, hormones and hormone analogs (e.g., growth hormone, adrenocorticotropic hormone, luteinizing hormone releasing hormone (LHRH), GLP-1 and exendin), vaccines (e.g., tumoral, bacterial and viral antigens), antigens, blood coagulation factors; growth factors; peptides such as protein inhibitors, protein antagonists, and protein agonists; nucleic acids, such as antisense molecules; oligonucleotides; ribozymes and derivatives (e.g., pegylated derivatives) thereof.

As defined herein, a sustained release of biologically active agent is a release of the agent from the sustained release composition of the invention which occurs over a period which is longer than that period during which a biologically significant amount of the active agent would be available following direct administration of a solution of the active agent. It is preferred that a sustained release be a release which occurs over a period of at least about a few days, for example, about one week, about two weeks, about three weeks or about four weeks. The sustained release can be a continuous or a discontinuous release, with relatively constant or varying rates of release. The continuity of release and level of release can be affected by the type of base polymer used, the type and amount of viscosity reducing agent selected, the active agent loading, and/or selection of excipients to produce the desired effect.

As used herein, a therapeutically effective amount, prophylactically effective amount or diagnostically effective amount is the amount of the sustained release composition needed to elicit the desired biological response following administration.

The sustained release compositions prepared according to the invention can contain from about 0.01 g to about 90 g of the biologically active agent per 100 g of polymer paste. g active agent/100 g polymer paste refers to the weight of drug present if combined with 100 g of polymer paste to form the composition. For example, the drug is present at 20 g/100 g of polymer paste when 20 g of drug is added to 100 g of polymer paste. The amount of agent can vary depending upon the desired effect of the agent, the planned release levels, and the time span over which the agent is to be released. A preferred range of agent loading is about 0.1 g active agent/100 g polymer paste to about 75 g active agent/100 g polymer paste, for example, about 0.1 g active agent/100 g polymer paste to about 60 g active agent/100 g polymer paste. A more preferred range of agent loading is about 0.5 g active agent/100 g polymer paste to about 75 g of active agent/100 g polymer paste, for example, about 0.5 g active agent/100 g polymer paste to about 60 g active agent/100 g polymer paste. For example, the drug can be present at about 1 g, 10, 20, 30, 40, 50 or about 60 g per 100 g polymer paste.

In one embodiment, the biologically active agent is stabilized. The biologically active agent can be stabilized against degradation, loss of potency and/or loss of biological activity, all of which can occur during formation of the sustained release composition having the biologically active agent dispersed therein, and/or prior to and during in vivo release of the biologically active agent. In one embodiment, stabilization can result in a decrease in the solubility of the biologically active agent, the consequence of which is a reduction in the initial release of biologically active agent. In addition, the period of release of the biologically active agent can be prolonged.

Stabilization of the biologically active agent can be accomplished, for example, by the use of a stabilizing agent or a specific combination of stabilizing agents. “Stabilizing agent,” as that term is used herein, is any agent which binds or interacts in a covalent or non-covalent manner or is included with the biologically active agent. Stabilizing agents suitable for use in the invention are described in U.S. Pat. Nos. 5,716,644 and 5,674,534 to Zale, et al.; U.S. Pat. Nos. 5,654,010 and 5,667,808 to Johnson, et al.; U.S. Pat. No. 5,711,968 to Tracy, et al., and U.S. Pat. No. 6,265,389 to Burke, et al.; and in copending U.S. patent application Ser. No. 08/934,830 by Burke, et al., filed on Sep. 22, 1997, the entire teachings of each of which are incorporated herein by reference.

For example, a metal cation can be complexed with the biologically active agent, or the biologically active agent can be complexed with a polycationic complexing agent such as protamine, albumin, spermidine and spermine, or associated with a “salting-out” salt. In addition, a specific combination of stabilizing agents and/or excipients may be needed to optimize stabilization of the biologically active agent. For example, when the biologically active agent in the sustained release composition is an acid-stable or free sulfhydryl-containing protein such as β-IFN, a particular combination of stabilizing agents which includes a disaccharide and an acidic excipient can be added to the mixture. This type of stabilizing formulation is described in detail in U.S. Pat. No. 6,465,425 issued to Tracy, et al., on Oct. 15, 2002, the entire contents of which is incorporated herein by reference.

Suitable metal cations include any metal cation capable of complexing with the biologically active agent. A metal cation-stabilized biologically active agent, as defined herein, comprises a biologically active agent and at least one type of metal cation wherein the cation is not significantly oxidizing to the biologically active agent. In a particular embodiment, the metal cation is multivalent, for example, having a valency of +2 or more. If the agent is metal cation-stabilized, it is preferred that the metal cation is complexed to the biologically active agent.

Suitable stabilizing metal cations include biocompatible metal cations. A metal cation is biocompatible if the cation is non-toxic to the recipient in the quantities used and also presents no significant deleterious or untoward effects on the recipient's body such as a significant immunological reaction at the injection site. The suitability of metal cations for stabilizing biologically active agents and the ratio of metal cation to biologically active agent needed can be determined by one of ordinary skill in the art by performing a variety of stability indicating techniques such as polyacrylamide gel electrophoresis, isoelectric focusing, reverse phase chromatography, and High Performance Liquid Chromatography (HPLC) analysis on particles of metal cation-stabilized biologically active agents prior to and following particle size reduction and/or encapsulation. The molar ratio of metal cation to biologically active agent is typically between about 1:2 and about 100:1, preferably between about 2:1 and about 12:1.

Examples of stabilizing metal cations include, but are not limited to, K⁺, Zn⁺², Mg⁺² and Ca⁺². Stabilizing metal cations also include cations of transition metals such as Cu⁺². Combinations of metal cations can also be employed.

The biologically active agent can also be stabilized with at least one polycationic complexing agent. Suitable polycationic complexing agents include, but are not limited to, protamine, spermine, spermidine and albumin. The suitability of polycationic complexing agents for stabilizing biologically active agents can be determined by one of ordinary skill in the art in the manner described above for stabilization with a metal cation. An equal weight ratio of polycationic complexing agent to biologically active agent is suitable.

Further, excipients can be added to the compositions of the present invention such as, for example, to maintain the potency of the biologically active agent over the duration of release and to modify polymer degradation. One or more excipients can be added to the mixture which is then used to form the polymer/biologically active agent matrix. For example, an excipient may be suspended or dissolved along with polymer and agent in a solvent system prior to formation of the polymer drug matrix.

Suitable excipients include, for example, carbohydrates, amino acids, fatty acids, surfactants, and bulking agents. Such excipients are known to those of ordinary skill in the art. An acidic or a basic excipient is also suitable. The amount of excipient used is based on its ratio to the biologically active agent, on a weight basis. For amino acids, fatty acids and carbohydrates, such as sucrose, trehalose, lactose, mannitol, dextran and heparin, the ratio of carbohydrate to biologically active agent, is typically between about 1:10 and about 20:1. For surfactants, the ratio of surfactant to biologically active agent is typically between about 1:1000 and about 2:1. Bulking agents typically comprise inert materials. Suitable bulking agents are known to those of ordinary skill in the art.

The excipient can comprise a metal cation component which is separately dispersed within the sustained release composition. This metal cation component acts to modulate the release of the biologically active agent and is not complexed with the biologically active agent. The metal cation component can optionally contain the same species of metal cation, as is contained in the metal cation stabilized biologically active agent, if present, and/or can contain one or more different species of metal cation. The metal cation component acts to modulate the release of the biologically active agent from the polymer matrix of the sustained release composition and can enhance the stability of the biologically active agent in the composition. A metal cation component used in modulating release typically comprises at least one type of multivalent metal cation. Examples of metal cation components suitable to modulate release include or contain, for example, Mg(OH)₂, MgCO₃ (such as 4MgCO₃.Mg(OH)₂.5H₂O), MgSO₄, Zn(OAc)₂, Mg(OAc)₂, ZnCO₃ (such as 3Zn(OH)₂.2ZnCO₃)ZnSO₄, ZnCl₂, MgCl₂, CaCO₃, Zn₃(C₆H₅O₇)₂ and Mg₃(C₆H₅O₇)₂. A suitable ratio of metal cation component to polymer is between about 1:99 to about 1:2 by weight. The optimum ratio depends upon the polymer and the metal cation component utilized and can be determined by one of ordinary skill in the art without undue experimentation. A polymer matrix containing a dispersed metal cation component to modulate the release of a biologically active agent from the polymer matrix is further described in U.S. Pat. No. 5,656,297 issued to Bernstein, et al., on Aug. 12, 1997, and U.S. Pat. No. 5,912,015 issued to Bernstein, et al., on Jun. 15, 1999, the entire contents of both of which are incorporated herein by reference.

The composition of this invention can be administered in vivo, for example, to a human or to an animal by injection, implantation (e.g., subcutaneously, intramuscularly, intraperitoneally, intracranially, and intradermally), administration to mucosal membranes (e.g., intranasally, intravaginally, intrapulmonary, buccally or by means of a suppository), topically, or in situ delivery (e.g., by enema or aerosol spray) to provide the desired dosage of biologically active agent based on the known parameters for treatment with the particular agent of the various medical conditions. It is preferred that the sustained release composition of the present invention is injected.

The sustained release composition can be administered using any dosing schedule which achieves the desired therapeutic levels for the desired period of time. For example, the sustained release composition can be administered and the patient monitored until levels of the drug being delivered return to baseline. Following a return to baseline, the sustained release composition can be administered again. Alternatively, the subsequent administration of the sustained release composition can occur prior to achieving baseline levels in the patient.

In some instances, it can be desirable to heat the sustained release composition to approximately body temperature prior to administration. The increase in temperature prior to injection can reduce the viscosity of the sustained release composition providing increased flowability and ease of administration. Syringes with heating mechanisms are known and can be suitably adapted, if needed, to administer the sustained release composition described herein.

The injectable sustained release compositions of the present invention are used in methods for providing therapeutically, prophylactically, or diagnostically effective amounts of a biologically active agent to a subject for a sustained period. The injectable sustained release compositions described herein provide increased therapeutic benefits by reducing fluctuations in active agent concentration in blood, by providing a more desirable release profile and by potentially lowering the total amount of biologically active agent needed to provide a therapeutic benefit.

As used herein, a “therapeutically effective amount,” “prophylactically effective amount,” or “diagnostically effective amount” is the amount of the biologically active agent or of the sustained release composition of biologically active agent needed to elicit the desired biological, prophylactic or diagnostic response following administration.

“Patient” as that term is used herein refers to the recipient of the treatment. Mammalian and non-mammalian patients are included. In a specific embodiment, the patient is a mammal, such as a human, canine, murine, feline, bovine, ovine, swine or caprine. In a preferred embodiment, the patient is a human.

As used herein, the terms “a” and “an” refer to one or more.

Experimental

Preparation of Base Polymer

A number of different ultralow molecular weight base polymers were prepared and combined with a variety of viscosity reducing agents to determine suitable combinations for use in the sustained release compositions of the invention.

Viscosity

Viscosity, as used herein, is a measure of the resistance of a material to flow and equals shear stress divided by shear rate. The centipoise (cP) is the unit of measurement for viscosity. The viscosity measurements reported herein for the polymer pastes and sustained release compositions (comprising polymer paste and active agent) were conducted at 37° C.

The viscosity of the base polymer/viscosity reducing agent combination, referred to herein as the polymer paste, and of the sustained release composition (polymer paste and drug) was measured using a Brookfield viscometer (cone and plate mechanism) at various temperatures and shear stresses. A Brookfield DV-II+ viscometer was used for measuring the viscosity of the prepared polymer paste. Spindle CP-52 was chosen, allowing viscosity measurements from 9.3 to 30,750 cP. The volume of each sample for the viscosity measurement was about 0.5 mL. The temperature of the sample was maintained at 37° C. using a controlled temperature water bath. The operating conditions of the DV-II+ viscometer were Torque(%): 10-90, Rotation Frequency (RPM): 2-60, Shear Stress (Pa): 2-855 and Temperature (° C.): 37.

A suitable viscosity for the polymer paste and the sustained release composition can be about 400 cP or less. A viscosity of about 400 cP or less can provide a composition suitable for injection. For example, the viscosity of the sustained release composition can be about 400 cP or less, such as about 300 cP or less, about 200 cP or less, about 100 cP or less, or about 50 cP or less.

Preparation of Polymer Pastes

### EXAMPLE 1

### Preparation of Base Polymers

Six different lactide/glycolide block copolymers with an inherent viscosity of about 0.12 dL/g or less were prepared using various proportions of the monomer units and a variety of initiators. The reaction scheme for polymer preparation is shown below along with a brief description of each polymer prepared:

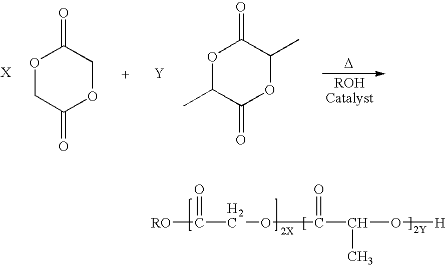

Polymerization Procedure

The base polymers described herein can be prepared using suitable methods known in the art. A representative synthesis is set forth below. It is understood that modifications in reaction conditions and reagents can be made to the representative synthesis. For example, reaction time and temperature can be modified to achieve the desired polymer. In addition, other suitable chain regulating agents, solvents and monomers can be employed.

ULMPLGA5050-1L

In a 500 mL flask equipped with a stirrer paddle, stirrer motor and gas outlet, 120.0 g of d,l-lactide and 96.0 g of glycolide were placed under nitrogen blanket. The reaction flask was purged with dry nitrogen by evacuating and releasing the vacuum five times. The flask and its contents were lowered into a silicon oil bath preheated at 170° C. (ULMPLGA5050-2L, prepared using same procedure but at a reaction temperature of 150° C).

After the monomers completely melted, 56.25 g of lauryl alcohol and 70.0 mg of stannous octoate were added. Eighteen hours later, vacuum was applied for about 2 hours to remove the unreacted monomers. The resulting polymer was collected by extruding into liquid nitrogen and immediately transferred into an amber jar. The yield of the copolymer obtained was 244.0 g (89.6%). The inherent viscosity of this copolymer was 0.05 dL/g measured in chloroform at 30° C.

TEGPLGA5050 (Tetraethylene glycol poly(lactide-co-glycolide))

Initiator: Tetraethylene glycol (‘TEG’ at the beginning of the nomenclature)

Composition: 50%/50% of lactide/glycolide monomer units in copolymer.

ULMPLGA5050-1L (Ultralow molecular weight poly(lactide-co-glycolide))

Initiator: Lauryl alcohol (‘L’ at the end of the nomenclature). ‘1’ represents version 1 (reaction temperature 170° C).

Composition: 50%/50% of lactide/glycolide monomer units in copolymer.

ULMPLGA5050-2L (Ultralow molecular weight poly(lactide-co-glycolide))

Initiator: Lauryl alcohol (‘L’ at the end of the nomenclature). ‘2’ represents version 2 (reaction temperature 150° C.).

Composition: 50%/50% of lactide/glycolide monomer units in copolymer.

ULMPLGA5050-1LA (Ultralow molecular weight poly(lactide-co-glycolide))

Initiator: Lauric Acid (‘LA’ at the end of the nomenclature). ‘1’ represents version 1.

Composition: 50%/50% of lactide/glycolide monomer units in copolymer.

ULMPLGA7525-1L (Ultralow molecular weight poly(lactide-co-glycolide))

Initiator: Lauryl alcohol (‘L’ at the end of the nomenclature). ‘1’ represents version 1.

Composition: 75%/25% of lactide/glycolide monomer units in copolymer.

ULMPDLLA-1L (Ultralow molecular weight poly((D,L-)lactide))

Initiator: Lauryl alcohol (‘L’ at the end of the nomenclature). ‘1’ represents version 1.

Composition: 100% lactide monomer units in polymer.

The polymer properties mass-average molecular weight, polydispersity index and inherent viscosity are listed in Table 1.

Inherent Viscosity

The inherent viscosities of the base polymers used to prepare the polymer pastes and sustained release compositions described herein, were determined using a polymer solution having 500 mg (±5 mg) dissolved to a total volume of 100 mL in chloroform. The measurements needed to calculate the inherent viscosity were conducted at 37° C.

First, the solvent efflux time (tS) was measure at 30° C. using a Cannon-Fenske Type Viscometer having a suitable capillary size, for example 25. The viscometer was used in accordance with manufacturer's instructions. Briefly, the tS was measured by drawing the solvent into the upper reservoir of the viscometer and allowing the solvent to drain through the capillary and measuring the time required for the solvent to pass between the graduation marks on the viscometer.

The efflux time of the polymer solution (tP) was similarly measured using the prepared polymer solution.

The relative viscosity of each polymer sample was then calculated as shown below:

Relative Viscosity (RV)=tS/tP

where:

tP=efflux time for the polymer sample (sec.)

tS=average efflux time for the solvent (sec.)

The inherent viscosity can be determined using the following calculation:

Inherent Viscosity (dL/g)=ln(RV)*1000*1/w=ln(RV)*1000/w

ln(RV)=natural logarithm of the relative viscosity

w=weight of polymer in the sample solution (mg)

TABLE 1

| | | Inherent | Average | Poly- |
|---|---|---|---|---|
| | | Visco- | Molecular | dispersity |
| | | sity | Weight | Index |
| Polymer | Initiator | (dL/g) | (Daltons) | (M_{w}/Mₙ) |
| TEGPLGA5050 | TEG | 0.05 | 1,090 | 1.28 |
| ULMPLGA5050-1L | Lauryl Alcohol | 0.05 | 2,120 | 1.51 |
| ULMPLGA5050-1LA | Lauryl Acid | 0.08 | 2,160 | 2.06 |
| ULMPLGA7525-1L | Lauryl Alcohol | 0.06 | 2,100 | 1.29 |
| ULMPDLLA-1L | Lauryl Alcohol | 0.05 | 2,070 | 1.28 |

### EXAMPLE 2

The appropriate polymer and viscosity reducing agent were each weighed into separate 3 mL syringes. Mixing of the polymer and viscosity reducing agent was conducted by connecting the syringes with a steel connector, and mixing the contents of the syringes until a polymer paste with a uniform appearance was achieved.

Polymer pastes with different proportions of polymer to additive were prepared and incubated in a buffer to study their degradation rate, which was recorded by measuring the weight loss over a period of time. Specifically, the following formulations were prepared and degradation measured:

ULMPLGA-1LA+40 WT. % PEG300

ULMPDLLA+40 WT. % PEG300

ULMPDLLA+20 WT. % PEG 300

ULMPDLLA (NO PEG)

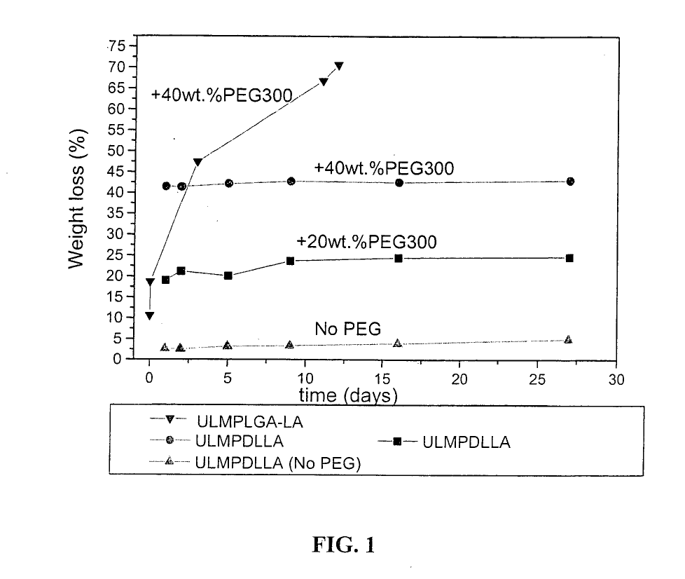

The results of the degradation study are shown in FIG. 1. FIG. 1 shows that for the ULMPDLLA polymer/PEG mixtures, weight loss over the first few days corresponds to the weight of the additive and that thereafter there is no significant weight loss. However, FIG. 1 shows that in the case of ULMPLGA+40 wt % PEG300, the degradation continues at a significant rate. In view of the above, formulations having the desired physical properties can be prepared by suitable choice of the base polymer.

### EXAMPLE 3

### Assessment of Viscosity Reducing Agents

The TEGPLGA polymer prepared in Example 1 was combined with the following viscosity reducing agents: acetic acid, DMSO, PEG 200, lactic acid, aqueous microsphere diluent (3 wt % carboxymethyl cellulose (CMC), 0.1 wt % TWEEN® 20 and 0.9 wt % NaCl), PEG 400, MPEG 350, polysorbate 80, PLURONIC® L101, PLURONIC® L64 and PLURONIC® L31 to form a polymer paste and the viscosity of the resulting polymer paste was then determined.

Specifically, the TEGPLGA and each viscosity reducing agent were combined at a 60/40 wt % ratio (60 wt % TEGPLGA and 40 wt % viscosity reducing agent). Briefly, the appropriate amount of TEGPLGA and viscosity reducing agent were weighed into separate 3 mL syringes. Mixing of the polymer and viscosity reducing agent was conducted by connecting the syringes with a steel connector, and mixing the contents of the syringes until a polymer paste with a uniform appearance was achieved.

A Brookfield DV-II+ viscometer was used for measuring the viscosity of the prepared polymer paste. Spindle CP-52 was chosen, allowing viscosity measurements from 9.3 to 30,750 cP. The volume of each sample for the viscosity measurement was about 0.5 mL. The temperature of the sample was maintained at 37° C. using a controlled temperature water bath. The operating conditions of the DV-II+ viscometer were Torque(%): 10-90, Rotation Frequency (RPM): 2-60, Shear Stress (Pa): 2-855 and Temperature (° C): 37.

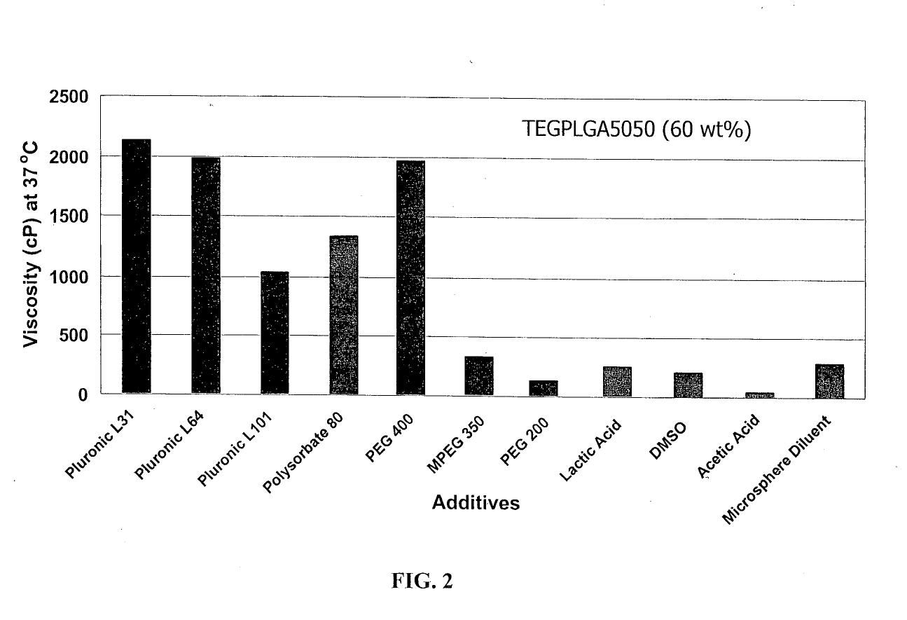

The results of the viscosity measurements of the prepared polymer pastes are shown in FIG. 2.

### EXAMPLE 4

### Assessment of Base Polymers

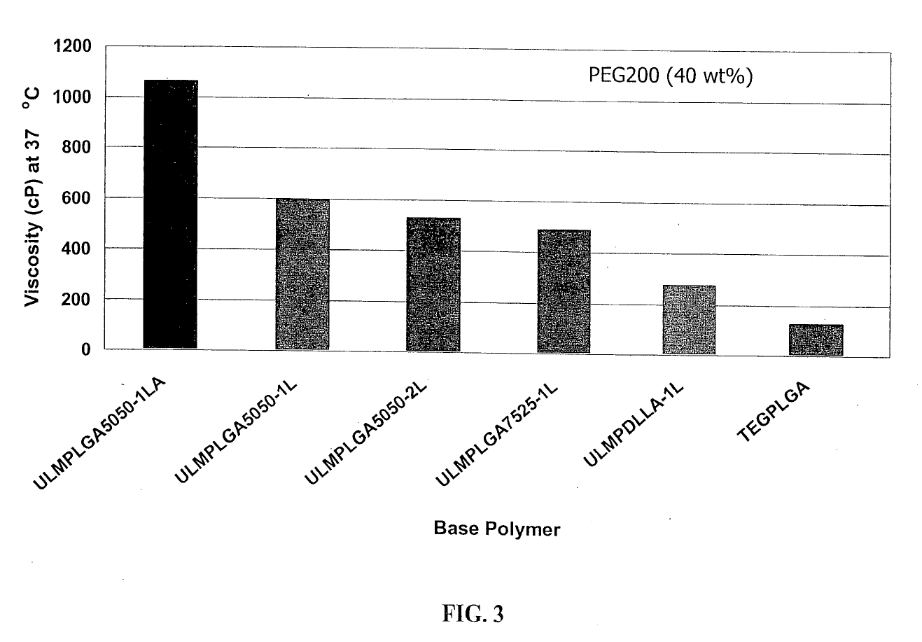

Each of the base polymers prepared in Example 1 were combined with the viscosity reducing agent, PEG200 to form a polymer paste having a 60/40 wt % ratio of base polymer to PEG 200. The polymer pastes were prepared as described in Example 3. The viscosity of each polymer paste was determined as described in Example 3 at 37° C. The results are depicted graphically in FIG. 3. Based on these results, further experiments were conducted using the ULMPDLLA and UMLPLGA5050-2L polymers.

### EXAMPLE 5

### Preparation and Assessment of Polymer Pastes

Specific combinations of base polymer and viscosity reducing agent were prepared and the viscosity of the resulting polymer pastes determined. Briefly, polymer pastes having a base polymer to viscosity reducing agent ratio of 60/40 wt % were prepared as described in Example 3. The specific combinations of base polymer and viscosity reducing agent are set forth in Table 2.

TABLE 2

| PASTE | BASE POLYMER | VISCOSITY REDUCING |
|---|---|---|
| FORMULATION | (at 60 wt %) | AGENT (at 40 wt %) |
| P-1 | TEGPLGA | PEG200 |
| P-2 | TEGPLGA | Acetic Acid |
| P-3 | TEGPLGA | DMSO |
| P-4 | ULMPDLLA-1L | PEG200 |
| P-5 | ULMPDLLA-1L | Acetic Acid |
| P-6 | ULMPDLLA-1L | DMSO |
| P-7 | ULMPLGA5050-2L | PEG200 |
| P-8 | ULMPLGA5050-2L | Acetic Acid |
| P-9 | ULMPLGA5050-2L | DMSO |

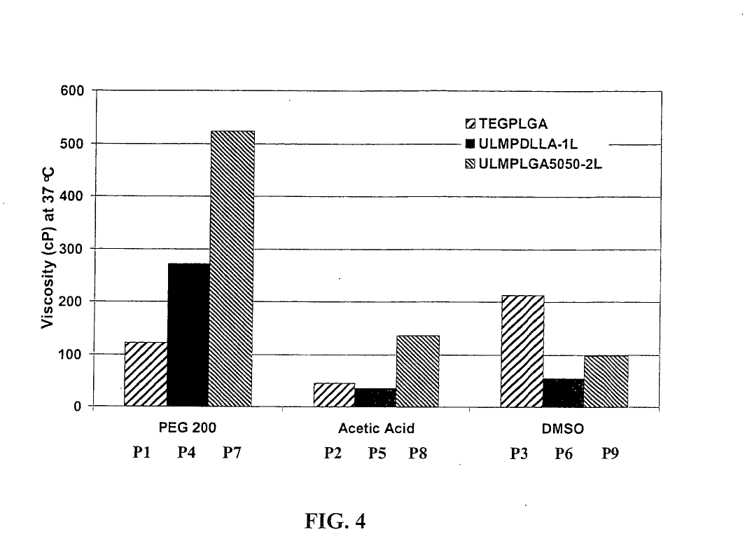

The viscosity was determined at 37° C. as described in Example 3. The results of viscosity testing on the paste formulations P1-P9 are depicted graphically in FIG. 4.

### EXAMPLE 6

### Assessment of Polymer Concentration

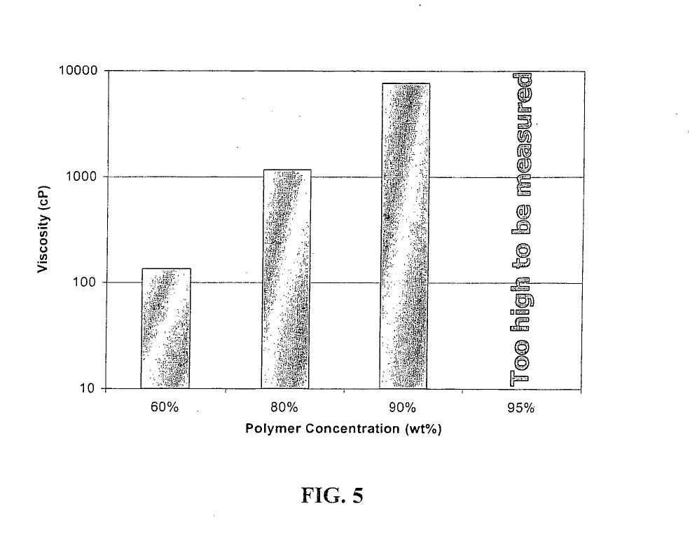

Selected combinations of the base polymer, ULMPLGA5050-2L with acetic acid as the viscosity reducing agent were prepared varying the ratio of base polymer to viscosity reducing agent. Briefly, polymer pastes having a ULMPLGA5050-2L to acetic acid ratio of 60/40 wt %, 80/20 wt %, 90/10 wt % and 95/5 wt % were prepared as described in Example 3. The viscosity of each combination was determined at 37° C. as described in Example 3. The results are depicted graphically in FIG. 5.

### EXAMPLE 7

### Combinations of Viscosity Reducing Agents

Various combinations of viscosity reducing agents were assessed for their ability to provide a polymer paste having a desired viscosity. Selected combinations of base polymer and viscosity reducing agent were prepared and the viscosity of the resulting polymer pastes determined. Briefly, polymer pastes having a base polymer to viscosity reducing agent ratio of 60/40 wt % were prepared as described in Example 3. A description of each polymer paste is set forth in Table 3.

TABLE 3

| PASTE | | |
|---|---|---|
| FORMULATION | BASE POLYMER | |
| AGENTS | (at 60 wt %) | VISCOSITY REDUCING |
| P-10 | TEGPLGA | PEG200 (20 wt %) + DMSO |
| | | (20 wt %) |
| P-11 | TEGPLGA | PEG200 (20 wt %) + Acetic |
| | | Acid (20 wt %) |
| P-12 | TEGPLGA | DMSO (20 wt %) + Acetic |
| | | Acid (20 wt %) |
| P-13 | TEGPLGA | PEG200 (20 wt %) + DMSO |
| | | (10 wt %) + Acetic |
| | | Acid (10 wt %) |
| P-14 | ULMPDLLA-1L | PEG200 (20 wt %) + DMSO |
| | | (20 wt %) |
| P-15 | ULMPDLLA-1L | PEG200 (20 wt %) + Acetic |
| | | Acid (20 wt %) |
| P-16 | ULMPDLLA-1L | DMSO (20 wt %) + Acetic |
| | | Acid (20 wt %) |
| P-17 | ULMPDLLA-1L | PEG200 (20 wt %) + DMSO (10 |
| | | wt %) + Acetic Acid (10 wt %) |
| P-18 | ULMPLGA5050-2L | PEG200 (20 wt %) + DMSO |
| | | (20 wt %) |
| P-19 | ULMPLGA5050-2L | PEG200 (20 wt %) + Acetic |
| | | Acid (20 wt %) |
| P-20 | ULMPLGA5050-2L | DMSO (20 wt %) + Acetic |
| | | Acid (20 wt %) |
| P-21 | ULMPLGA5050-2L | PEG200 (20 wt %) + |
| | | DMSO (10 wt %) + |
| | | Acetic Acid (10 wt %) |

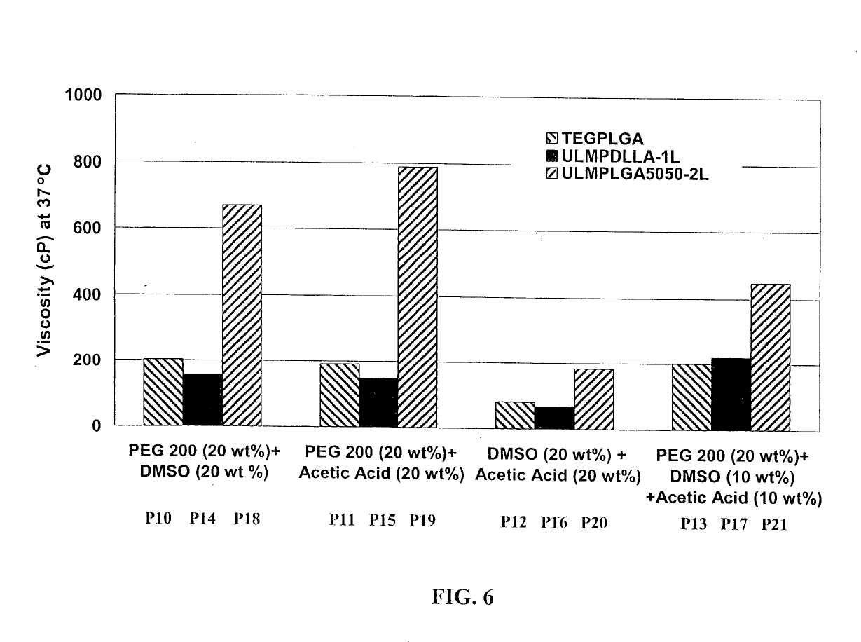

The viscosity was determined at 37° C. as described in Example 3. The results of viscosity testing on the polymer paste formulations P10-P21 are depicted graphically in FIG. 6.

Further combinations of viscosity reducing agents were assessed for their ability to provide a polymer paste having a desired viscosity. Selected combinations of base polymer and viscosity reducing agent were prepared and the viscosity of the resulting polymer pastes determined. Briefly, polymer pastes having a base polymer to viscosity reducing agent ratio of 60/40 wt % were prepared as described in Example 3. A description of each polymer paste and resulting viscosity is set forth in Table 4.

TABLE 4

| PASTE | | VISCOSITY |
|---|---|---|
| FORMULATION | | REDUCING |
| (VISCOSITY, cP) | BASE POLYMER | AGENTS |
| P-22 | ULMPDLLA-1L | PEG200 (30%) + DMSO |
| (215 cP) | | (5%) + Acetic |
| | | Acid (5%) |
| P-23 | ULMPDLLA-1L | DILUENT (30%) + DMSO |
| (454 cP) | | (5%) + ACETIC |
| | | ACID (5%) |

It is noted that use of diluent (3 wt % carboxymethyl cellulose (CMC), 0.1 wt % TWEEN® 20 and 0.9 wt % NaCl) in the P-23 polymer paste formulation results in a higher viscosity than that achieved in the P-22 formulation using PEG200. It is likely that the immiscibility of the vehicle is related to the increased viscosity.

### EXAMPLE 8

### Mixtures of Base Polymers

Base polymer mixtures were combined with selected viscosity reducing agents and assessed for their ability to provide a polymer paste having a desired viscosity. Selected mixtures of base polymer and viscosity reducing agent were prepared and the viscosity of the resulting polymer pastes determined. Briefly, polymer pastes having a base polymer mixture to viscosity reducing agent ratio of 60/40 wt % were prepared as described in Example 3. Polymer pastes containing each polymer of the polymer mixture alone were also prepared for comparison. A description of each polymer paste is set forth in Table 5.

TABLE 5

| | | VISCOSITY |
|---|---|---|
| PASTE | | REDUCING |
| FORMULATION | BASE POLYMER | AGENTS |
| P-24 | TEGPLGA (60%) | DMSO (20%) + ACETIC |
| | | ACID (20%) |
| P-25 | ULMPLGA5050-1LA | DMSO (20%) + ACETIC |
| | (30%) and | ACID (20%) |
| | TEGPLGA (30%) | |
| P-26 | ULMPLGA5050-1LA | DMSO (20%) + ACETIC |
| | (60%) | ACID (20%) |

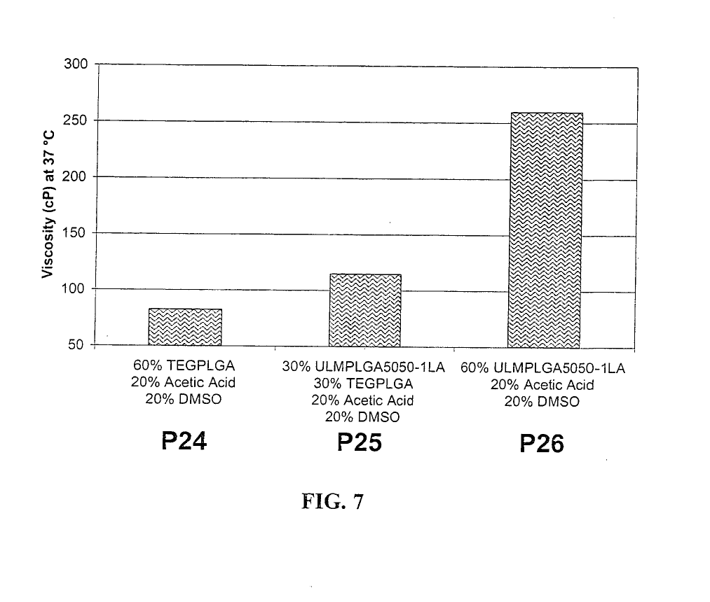

The results of viscosity testing are set forth graphically in FIG. 7. FIG. 7 shows that the viscosity of the polymer paste formulation using the polymer mixture is between the two polymers when each is used alone indicating the presence of other interactions among the polymer upon mixing.

### EXAMPLE 9

### Temperature Effects

The temperature effect on the viscosity was studied for paste formulations having TEGPLGA-1L as the base polymer and PEG200, MPEG350, aqueous diluent and lactic acid as the viscosity reducing agents. Temperature was controlled using a water bath connected to the Brookfield viscometer.

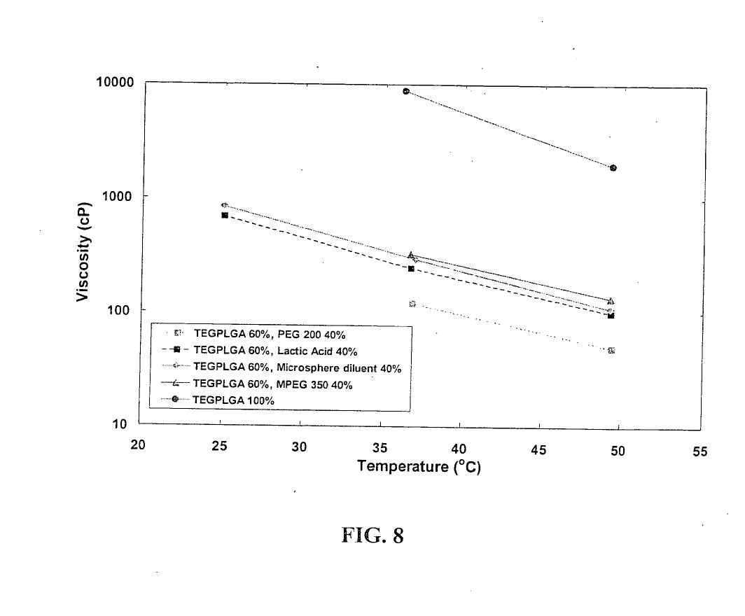

The result of the viscosity testing of the polymer paste formulations as well as the base polymer alone (TEGPLGA-1L) are depicted graphically in FIG. 8. Each point in the graph is an average of the measurements made at a given temperature and different shear stresses. The maximum standard deviation for the viscosity measured at different shear stresses was 4.5%. The results show that there is a significant relationship between the temperature and viscosity and that the viscosity is significantly greater for the polymer alone as compared to the polymer paste formulations prepared.

### EXAMPLE 10

### Shear Stress Effect

Typically polymers exhibit pseudoplastic behavior wherein the viscosity decreases with increasing shear resulting from alignment of the random coils of the polymer in the direction of the shear which reduces the resistance to flow and decreases the viscosity. The viscosity of certain polymer paste formulations was determined at different shear rates in order to assess the dependence of the polymer pastes on shear.

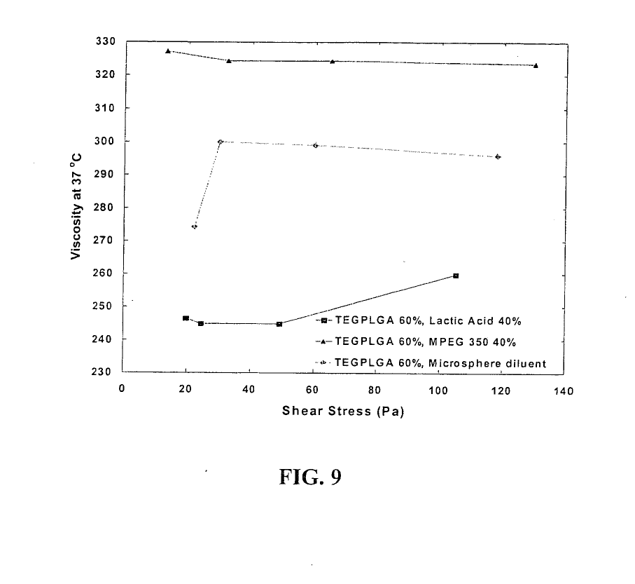

The base polymer was TEGPLGA (60%) and the viscosity reducing additives were lactic acid, MPEG250 or aqueous diluent, all present at a 40% concentration. The shear stress is displayed by the instrument as the frequency of rotation of the spindle in the viscometer within the suitable torque measurement range (10 to 90%) was varied. The results of shear stress effect are set forth graphically in FIG. 9.

FIG. 9 shows that higher shear stress generally leads to slightly lower viscosity. However, the effect of the shear is not significant since an increase of 100 Pa in shear stress leads to a decrease of about less than 10 cP in viscosity.

### EXAMPLE 11

### Effect of Drug Load on Viscosity of Polymer Paste

The effect of drug load on the viscosity of different polymer paste formulations was assessed. Naltrexone was mixed with the polymer paste formulation ULMPLGA5050-2L (60%)/PEG200 (40%) at both a 20 g/100 g paste and 50 g/100 g paste load. The viscosity of the polymer paste formulation with no drug incorporated was also determined. The results of viscosity testing are set forth in Table 6.

TABLE 6

| | NALTREXONE LOAD | VISCOSITY AT |
|---|---|---|
| PASTE FORMULATION | (g/100 g PASTE) | 37° C. (cP) |
| ULMPLGA5050-2L | 0 | 523 |
| (60%) + PEG200 (40%) | | |
| ULMPLGA5050-2L | 20 | 1130 |
| (60%) + PEG200 (40%) | | |
| ULMPLGA5050-2L | 50 | >10,000 |
| (60%) + PEG200 (40%) | | |

### EXAMPLE 12

### Effect of Additives on Viscosity at Constant Drug Load

Three polymer paste formulations having base polymer ULMPLGA5050-2L (60%) and either PEG200, DMSO or acetic acid at a concentration of 40% were prepared and loaded with naltrexone at a level of 20 g/100 g of polymer paste. The viscosity of the compositions were determined and the results are set forth in Table 7.

TABLE 7

| | VISCOSITY AT |
|---|---|
| SUSTAINED RELEASE COMPOSITION | 37° C. (cP) |
| ULMPLGA5050-2L (60%) + PEG200 | 1130 |
| (40%) + NALTREXONE (20 g/100 g | |
| PASTE) | |
| ULMPLGA5050-2L (60%) + DMSO (40%) + | 1645 |
| NALTREXONE (20 g/100 g PASTE) | |
| ULMPLGA5050-2L (60%) + ACETIC | 1189 |
| ACID (40%) + NALTREXONE | |
| (20 g/100 g PASTE) | |

Preparation of Sustained Release Injectable Compositions Drug Release Studies

Sustained release compositions comprising base polymer, viscosity reducing agent and drug were prepared, characterized and drug release assessed. The drugs which were incorporated into the various polymer pastes were naltrexone and insulin.

Insulin

### EXAMPLE 13

### Preparation of Insulin Containing Sustained Release Compositions

Sustained release compositions comprising insulin at a drug load of 1.2 g/100 g of polymer paste were prepared. The insulin was purchased from Disynth and further formulated into a powder containing a 30:1 molar ratio of zinc/insulin by the addition of zinc acetate to a solution of insulin, followed by spray freeze drying of the resulting mixture. The type and amount of base polymer and viscosity reducing agent in the polymer paste were varied for the sustained release compositions prepared. Briefly, about 0.5 mL of base polymer was weighed in a 3 mL syringe. The desired amount of viscosity reducing agent was determined by weight percent and weighed into a second syringe. The desired amount of insulin was calculated based on the total volume of the polymer and the viscosity reducing agent and added to the viscosity reducing agent to obtain a mixture of insulin and viscosity reducing agent. Polymer was then combined with the insulin/viscosity reducing agent mixture using a connector for the 3 mL syringes and mixing the contents of the syringes until a visually homogeneous paste was obtained. The sustained release compositions containing insulin are listed in Table 8.

TABLE 8

| SUSTAINED | | |
|---|---|---|
| RELEASE | BASE POLYMER | VISCOSITY REDUCING |
| COMPOSITION | (wt % OF PASTE) | AGENT (wt % OF PASTE) |
| I1 | ULMPLGA5050-2L | — |
| | (100%) | |
| I2 | ULMPLGA5050-2L | PEG200 (20%) |
| | (80%) | |
| I3 | ULMPLGA5050-2L | PEG200 (40%) |
| | (60%) | |
| I4 | — | PEG200 (100%) |
| I5 | ULMPLGA5050-2L | DMSO (20%) |
| | (80%) | |
| I6 | ULMPLGA5050-2L | DMSO (40%) |
| | (60%) | |
| I7 | ULMPLGA5050-2L | ACETIC ACID (20%) |
| | (80%) | |
| I8 | ULMPLGA5050-2L | ACETIC ACID (40%) |
| | (60%) | |
| I9 | ULMPLGA5050-2L | PEG200 (20%) + DMSO |
| | (60%) | (20%) |
| I10 | ULMPLGA5050-2L | PEG (20%) + ACETIC |
| | (60%) | ACID (20%) |
| I11 | ULMPLGA5050-2L | DMSO (20%) + ACETIC |
| | (60%) | ACID (20%) |
| I12 | ULMPLGA5050-2L | PEG200 (20%) + DMSO |
| | (60%) | (10%) + ACETIC ACID |
| | | (10%) |
| I13 | ULMPDLLA (60%) | PEG200 (40%) |
| I14 | ULMPDLLA (60%) | PEG200 (20%) + DMSO |
| | | (20%) |
| I15 | ULMPDLLA (60%) | PEG200 (20%) + ACETIC |
| | | ACID (20%) |
| I16 | ULMPDLLA (60%) | DMSO (20%) + ACETIC |
| | | ACID (20%) |
| I17 | ULMPDLLA (60%) | PEG200 (20%) + DMSO |
| | | (10%) + ACETIC ACID |
| | | (10%) |
| I18 | TEGPLGA (60%) | PEG200 (40%) |
| I19 | TEGPLGA (60%) | PEG200 (20%) + DMSO |
| | | (20%) |
| I20 | TEGPLGA (60%) | PEG200 (20%) + ACETIC |
| | | ACID (20%) |
| I21 | TEGPLGA (60%) | DMSO (20%) + ACETIC |
| | | ACID (20%) |
| I22 | TEGPLGA (60%) | PEG200 (20%) + DMSO |
| | | (10%) + ACETIC ACID |
| | | (10%) |
| I23 | ULMPLGA5050-LA | DMSO (20%) + ACETIC |
| | (60%) | ACID (20%) |
| I24 | ULMPLGA5050-LA | DMSO (20%) + ACETIC |
| | (30%) + TEGPLGA | ACID (20%) |
| | (30%) | |

### EXAMPLE 14

### Insulin Release

In vitro release experiments were conducted by incubating a predetermined amount of the sustained release compositions of Table 8 with a HEPES/KCl/NaN₃ buffer (HEPES (Na salt) 0.05 mol/L, KCl 0.01 mol/L and NaN₃ 0.1 g/100 mL adjusted to pH 7.03 with 0.1 M HCl). Briefly, 0.25 mL of sustained release composition was incubated with 1 mL of buffer at about 37° C. Sampling was conducted at predetermined time points by removing the complete amount of buffer (1 mL) and adding fresh buffer (1 mL). To study initial release, samples were taken following 18 hours of incubation. To determine cumulative release samples were taken at 24 hours intervals following incubation.

Prior to determining the amount of insulin present in the incubation buffer, the sample was filtered using a 0.2 micron syringe filter. The UV absorbance of the filtered sample at 280 nanometers was then obtained and the concentration of the insulin in the buffer determined using a UV calibration curve prepared for insulin at 280 nanometers.

The percentage of released insulin was determined by dividing the amount of insulin released in 1 mL of release buffer by the total amount of insulin in the starting polymer paste.

The cumulative percentage of insulin released was determined by adding the percentage released at the current sampling time point to the sum of the percentage released from all prior sampling time points.

### EXAMPLE 15

### Effect of Base Polymer on Insulin Release

A: ULMPLGA5050-2L, ULMPDLLA and TEGPLGA with PEG200:

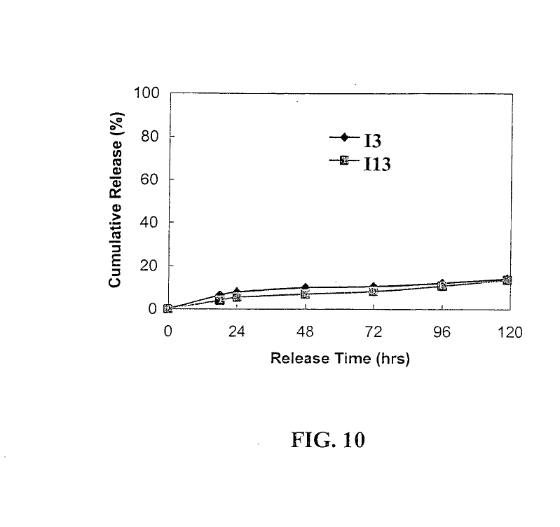

The effects of the different base polymers, ULMPLGA5050-2L, ULMPDLLA and TEGPLGA on drug release can be seen by comparing sample I3, I13 and I18 of Table 8. The three base polymers are all present at 60 wt % in the compositions and have 40 wt % of PEG200. Due to the dissolution of the TEGPLGA polymer in the release buffer, accurate measurements of insulin concentration in the incubation buffer could not be determined for this paste (I18). The cumulative release of insulin from compositions I3 and I13 are depicted graphically in FIG. 10. FIG. 10 shows that ULMPDLLA (I13) had lower initial release ˜4% than ULMPLGA5050-2L (I3) ˜7%.

B: ULMPLGA5050-2L and ULMPDLLA with DMSO (20%) and Acetic Acid (20%):

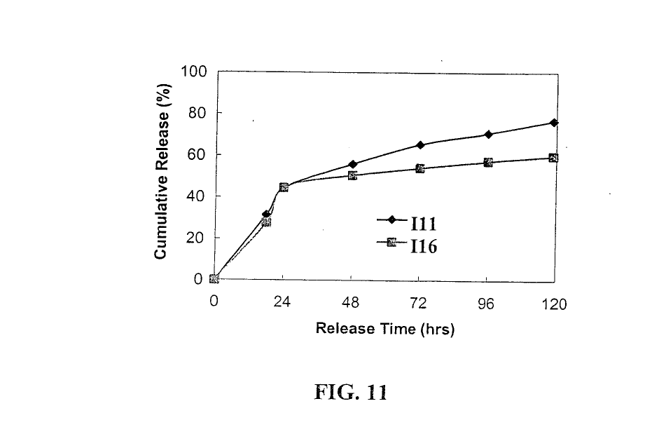

A comparison of cumulative % release of I11 and I16, ULMPLGA5050-2L and ULMPDLLA with DMSO (20%)+acetic acid (20%) respectively, is depicted graphically in FIG. 11. Comparison of the release shown in FIG. 10 with the release shown in FIG. 11 indicates that the DMSO/acetic acid combination results in a higher initial release than compositions prepared using PEG200 alone.

C: Combination of Base Polymers

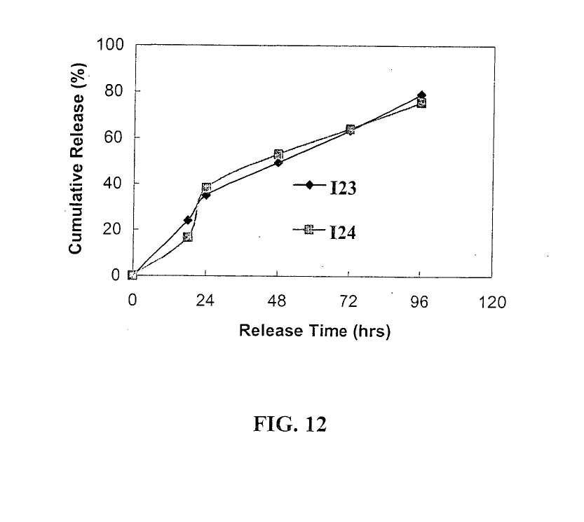

The cumulative release for compositions I23 (60 wt % ULMPLGA5050-LA+20% DMSO+20% acetic acid) and I24 (30 wt % ULMPLGA5050LA+30 wt % TEGPLGA+20% DMSO+20% acetic acid) were determined. The cumulative release is depicted graphically in FIG. 12. FIG. 12 shows that the cumulative release of insulin from the two compositions is similar.

### EXAMPLE 16

### Effect of Viscosity Reducing Agents

A: Comparison of PEG200, DMSO and Acetic Acid

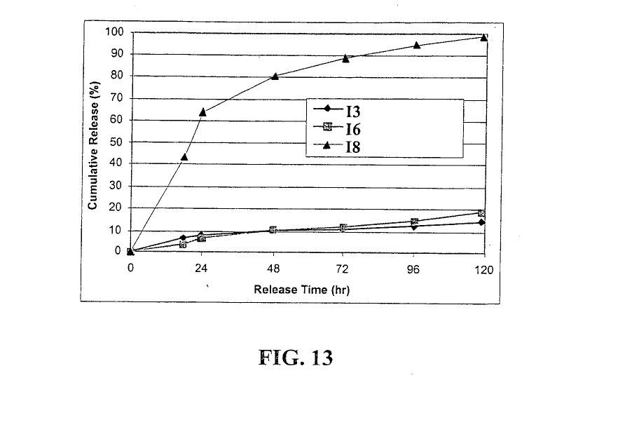

The release of insulin from compositions having 40% PEG200, DMSO or acetic acid with 60% ULMPLGA5050-2L (I3, I6 and I8 of Table 8, respectively) was determined and a comparison of the profiles are depicted graphically in FIG. 13. Acetic acid gave an initial release of about 45% at 18 hours with 100% of insulin released at five days. PEG 200 and DMSO gave initial releases similar to each other, with the amount released in subsequent 24 hour periods tested about 5% per day.

B: Comparison of Different Combination of Additives

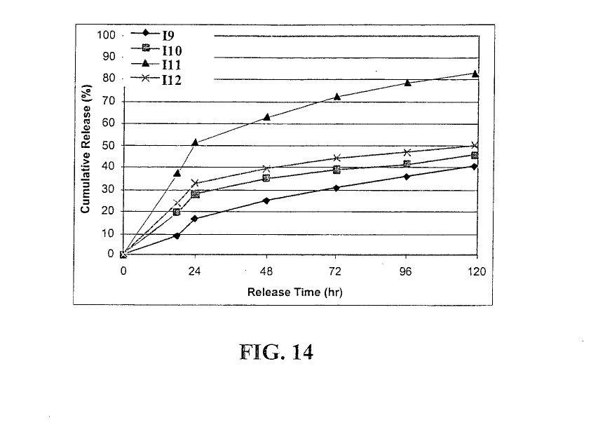

The release of insulin from compositions having ULMPLGA5050-2L with different combinations of additives is depicted graphically in FIG. 14. The compositions tested were I9 (20% PEG200+20% DMSO), I10 (20% PEG200+20% acetic acid), I11 (20% DMSO+20% acetic acid) and I12 (20% PEG200+10% DMSO+10% acetic acid) of Table 8. The graph shows that PEG200 provides a lower initial release than DMSO and acetic acid.

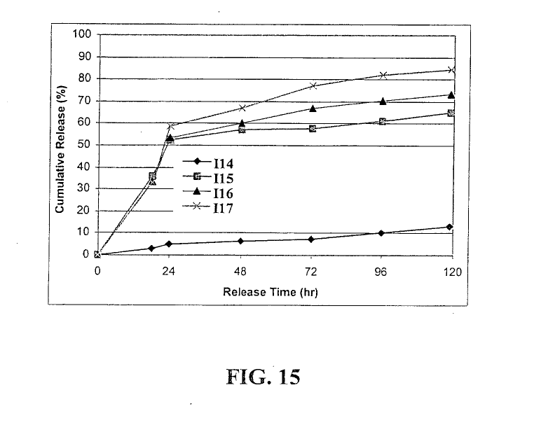

The same comparison as described above and depicted in FIG. 14 was conducted from the polymer ULMPDLLA (compositions I14, I15, I16 and I17 of Table 8). The results are depicted graphically in FIG. 15. Similar to the results of FIG. 14, the combination of DMSO and PEG200 provided the lowest initial release of insulin from the sustained release composition.

C: Effect of the Polymer to Viscosity Reducing Agent Ratio

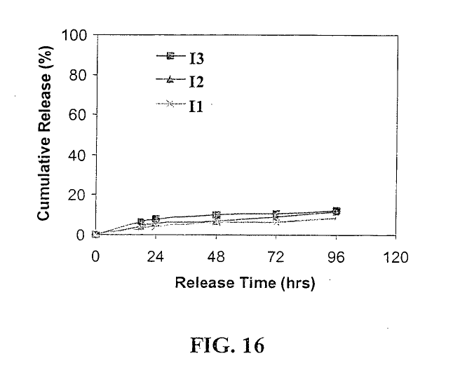

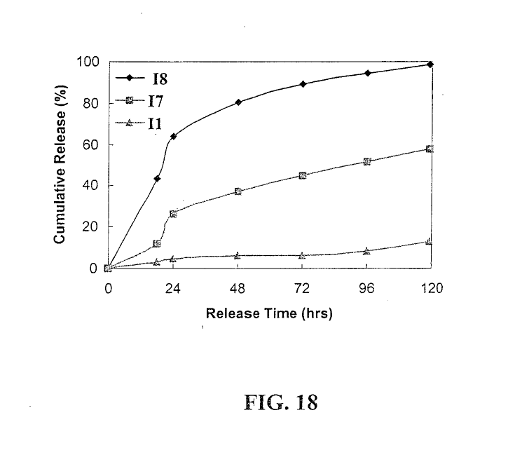

The cumulative release of insulin from sustained release compositions comprising ULMPLGA5050-2L with PEG200, DMSO and acetic acid at varying ratios of viscosity reducing agent were determined and are depicted graphically in FIGS. 16, 17 and 18, respectively.

FIG. 16 shows that when the concentration of viscosity reducing agent was 0 (I1) or 20% PEG200 (I2) the cumulative drug release was about 8% for five days. However, when the ratio was increased to 40% PEG200 (I3), the cumulative release increased to 12% in five days indicating that the greater the concentration of the base polymer the slower will be the release and that 60 wt % base polymer is a suitable amount.

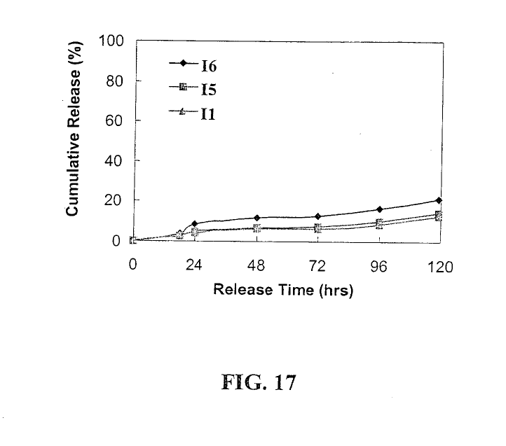

FIGS. 17 (I1, I5 and I6) and 18 (I1, I7 and I8) also show that the ratio of viscosity reducing agent to polymer effects the drug release profile with an increase in the agent providing an increased cumulative release.

Naltrexone

### EXAMPLE 17

### Preparation of Naltrexone Containing Sustained Release Compositions

Sustained release compositions comprising naltrexone at a drug load of 20 g/100 g of polymer paste and 50 g/100 g of polymer paste were prepared. The type and amount of base polymer and viscosity reducing agent in the polymer paste were varied for the sustained release compositions prepared. Briefly, the desired amount of base polymer was weighed in a 3 mL syringe. The desired amount of viscosity reducing agent was determined by weight percent and weighed into a second syringe. The desired amount of naltrexone was calculated based on the total volume of the polymer and the viscosity reducing agent and added to the viscosity reducing agent to obtain a mixture of naltrexone and viscosity reducing agent. The naltrexone/viscosity reducing agent mixture was sonicated. Polymer was then combined with the naltrexone/viscosity reducing agent mixture using a connector for the 3 mL syringes and mixing the contents of the syringes until a visually homogeneous polymer paste was obtained. The sustained release compositions containing naltrexone are listed in Table 9.

TABLE 9

| | | VISCOSITY | |
|---|---|---|---|
| SUSTAINED | | REDUCING | |
| RELEASE | BASE POLYMER | AGENT (% OF | NALTREXONE |
| COMPOSITION | (% OF PASTE) | PASTE) | (g/100 g PASTE) |
| N1 | ULMPLGA5050-2L (60%) | PEG200 (40%) | 50 |
| N2 | ULMPLGA5050-2L (60%) | PEG200 (40%) | 20 |
| N3 | ULMPLGA5050-2L | — | 20 |
| | (100%) | | |
| N4 | ULMPLGA5050-2L (80%) | PEG200 (20%) | 20 |
| N5 | ULMPLGA5050-2L (80%) | DMSO (20%) | 20 |
| N6 | ULMPLGA5050-2L (80%) | ACETIC ACID | |
| | | (20%) | |
| N7 | ULMPLGA5050-2L (60%) | DMSO (40%) | 20 |
| N8 | ULMPLGA5050-2L (60%) | ACETIC ACID | 20 |
| | | (40%) | |
| N9 | ULMPLGA5050-2L (60%) | PEG200 (20%) + DMSO | 20 |
| | | (20%) | |
| N10 | ULMPLGA5050-2L (60%) | PEG (20%) + ACETIC | 20 |
| | | ACID | |
| | | (20%) | |
| N11 | ULMPLGA5050-2L (60%) | DMSO (20%) + ACETIC | 20 |
| | | ACID | |
| | | (20%) | |
| N12 | ULMPDLLA (60%) | PEG200 (40%) | 20 |
| N13 | ULMPDLLA (60%) | PEG200 (20%) + DMSO | 20 |
| | | (20%) | |
| N14 | ULMPDLLA (60%) | PEG200 (20%) + ACETIC | 20 |
| | | ACID (20%) | |
| N15 | ULMPDLLA (60%) | DMSO (20%) + ACETIC | 20 |
| | | ACID | |
| | | (20%) | |
| N16 | TEGPLGA (60%) | PEG200 (40%) | 20 |
| N17 | TEGPLGA (60%) | PEG200 (20%) + DMSO | 20 |
| | | (20%) | |
| N18 | TEGPLGA (60%) | PEG200 (20%) + ACETIC | 20 |
| | | ACID (20%) | |
| N19 | TEGPLGA (60%) | DMSO (20%) + ACETIC | 20 |
| | | ACID | |
| | | (20%) | |
| N20 | ULMPLGA5050-LA | DMSO (20%) + ACETIC | 20 |
| | (60%) | ACID | |
| | | (20%) | |
| N21 | ULMPLGA5050-LA | DMSO (20%) + ACETIC | 20 |
| | (30%) + TEGPLGA (30%) | ACID (20%) | |

### EXAMPLE 18

### Naltrexone Release

In vitro release experiments were conducted by incubating a predetermined amount of the sustained release compositions of Table 9 with buffer (2.76 g monobasic, monohydrate sodium phosphate, 11.36 g of anhydrous dibasic sodium phosphate, 1.6 g of sodium chloride, 0.2 g of TWEEN® 20, 0.2 g of sodium azide in one liter of water; adjust pH to 7.4 with sodium hydroxide or phosphoric acid). Briefly, 0.25 mL of sustained release composition was incubated with 20 mL of buffer at about 37° C. Sampling was conducted at predetermined time points by removing buffer (4 mL) and adding fresh buffer (4 mL). To determine release, samples were analyzed by UV absorbance at 281 nanometers with background correction using 450 nanometers. A calibration curve of naltrexone in the release buffer (0.03-0.5 mg/mL) was used to determine an extinction coefficient of 3.7823 (mg/mL)⁻¹.

The percentage of released naltrexone was determined by dividing the concentration released by the concentration of the starting polymer paste.

The cumulative percentage of naltrexone released was determined by adding the percentage released at the current sampling time point to the sum of the percentage released from all prior sampling time points.

All results discussed below are the average of duplicate samplings.

### EXAMPLE 19

### Effect of Naltrexone Load

A: Effect of Polymer Paste on Naltrexone Release

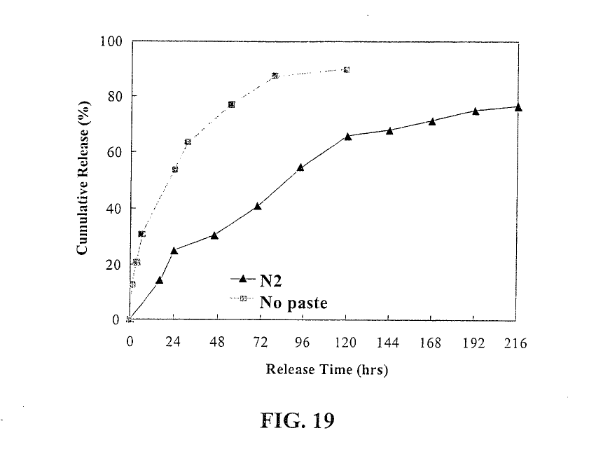

The effect of polymer paste on naltrexone release can be seen in FIG. 19 where the release profile for drug alone in buffer is compared to the release profile for composition N2 of Table 9. The mass of naltrexone was the same (50 micrograms). The release profiles show that the polymer paste having 60 wt % or polymer is suitable for sustaining the release of naltrexone.

B: Effect of Drug Load

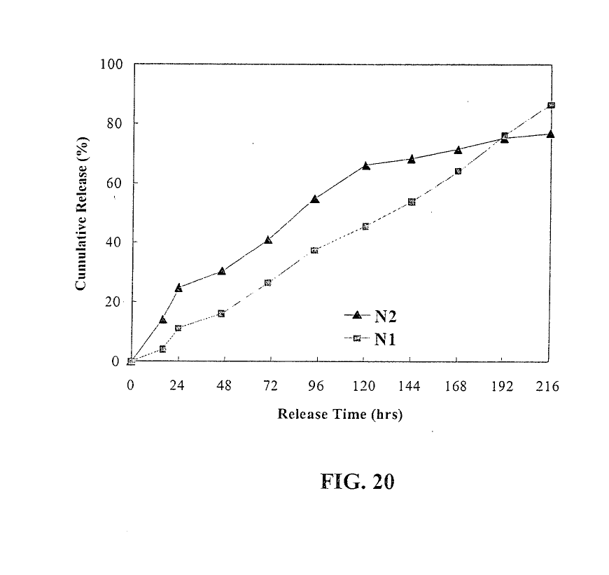

The release profiles for compositions N1 and N2 of Table 9 (60 wt % ULMPLGA5050-2L+40 wt % PEG200 and 50 g or 20 g of naltrexone, respectively) were determined. The release profiles are graphically depicted in FIG. 20.

FIG. 20 indicates that the relative % of drug released by the polymer paste with 50 g/100 g polymer paste drug load was similar to the % of drug released by the polymer paste with 20 g/100 g polymer paste drug load. After 120 hours the release rate from the paste with the 20 g of drug decreased, while the polymer paste with 50 g of drug continued to exhibit a constant release rate of about 10% per day. These results show that the polymer paste have the potential to deliver high drug loads without high initial release and with constant sustained release, particularly when the wt % of polymer in the polymer paste is about 60 wt % or more.

### EXAMPLE 20

### Effect of Base Polymer

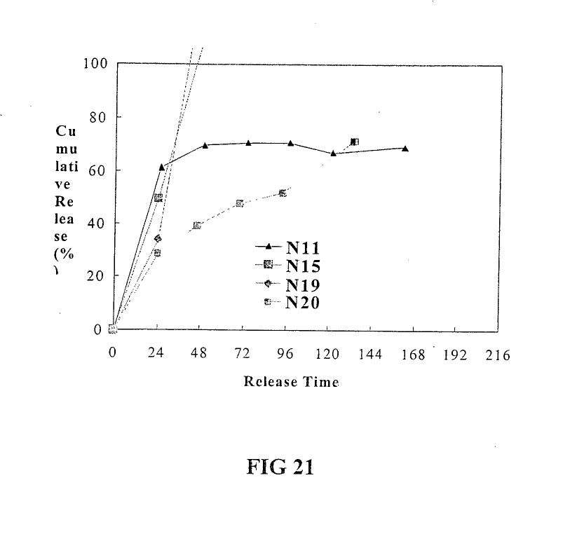

The effect of the base polymer on naltrexone release was assessed by comparing the release profiles of sustained release compositions wherein the drug load and viscosity reducing agents were held constant and base polymer varied. Comparison of the release profiles for compositions N11, N15, N19 and N20 of Table 9 is depicted graphically in FIG. 21. The viscosity reducing agent of each of the compositions of FIG. 21 was a mixture of 20 wt % DMSO+20 wt % acetic acid and the drug load was 20 g/100 g of polymer paste. The base polymer was varied as follows: N11 (ULMPLGA5050-2L), N15 (ULMPDLLA), N19 (TEGPLGA) and N20 (ULMPLGA5050-1 LA).

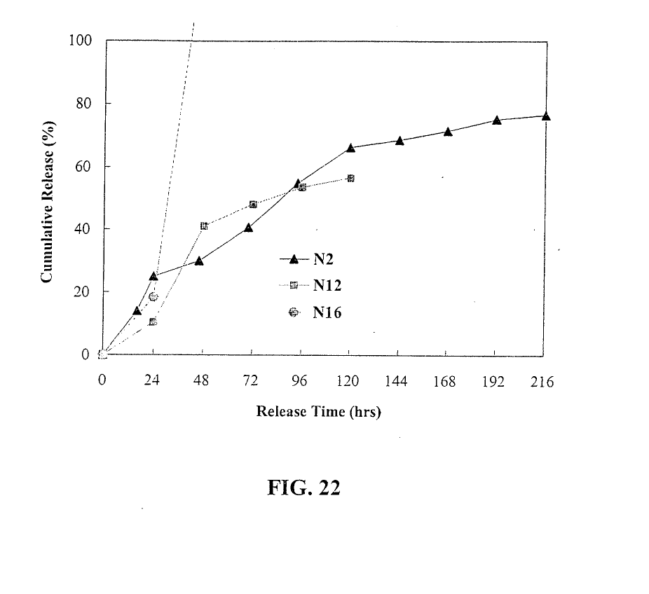

In a similar fashion, the release profiles of compositions N2, N12 and N16 each with a drug load of 20 g/100 g of polymer paste and 40% PEG200 as the viscosity reducing agent, but having ULMPLGA5050-2L, ULMPDLLA and TEGPLGA as the base polymers were determined and compared. A comparison of the release profiles for N2, N12 and N16 are depicted graphically in FIG. 22.

Comparison of the release profiles in FIGS. 21 and 22 show that the TEGPLGA provides a very fast release of drug. It is likely that this increased release is due to the affinity of the amphiphilic polymer for the aqueous incubation buffer. The release profiles for the compositions ULMPLGA5050-2L and ULMPDLLA-1L polymers appear to be similar.

### EXAMPLE 21

### Effect of Ratio of Base Polymer to Additive

The effect of varying the ratio of base polymer to additive were conducted using polymer pastes comprising ULMPLGA5050-2L as the base polymer and PEG200, DMSO or acetic acid as the viscosity reducing agents at a base polymer concentration of 100 wt %, 80 wt % and 60 wt %.

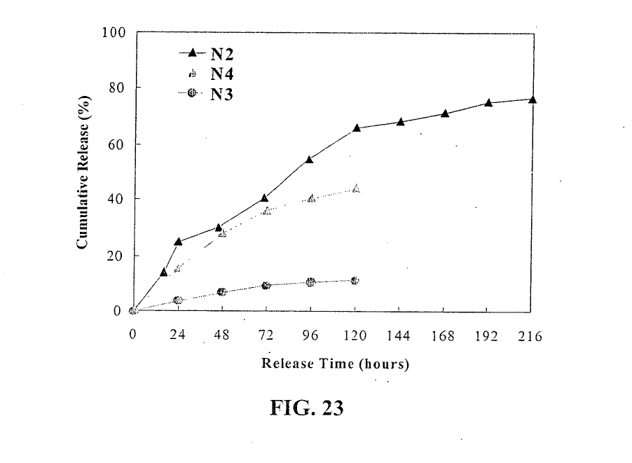

FIG. 23 shows the naltrexone release profiles of compositions having a 20 g/100 g polymer paste drug load and PEG200 as the viscosity reducing agent with a base polymer concentration in the polymer paste of 60% (N2), 100% (N3) and 80% (N4).

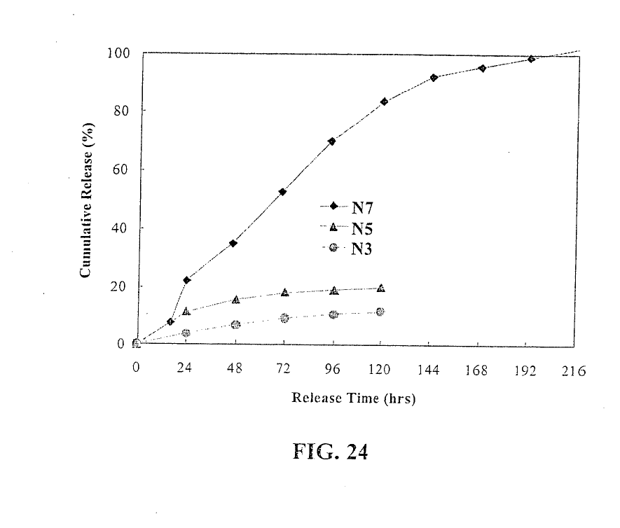

FIG. 24 shows the naltrexone release profiles of compositions having a 20 g/100 g polymer paste drug load and DMSO as the viscosity reducing agent with a base polymer concentration in the polymer paste of 60% (N7), 100% (N3) and 80% (N5).

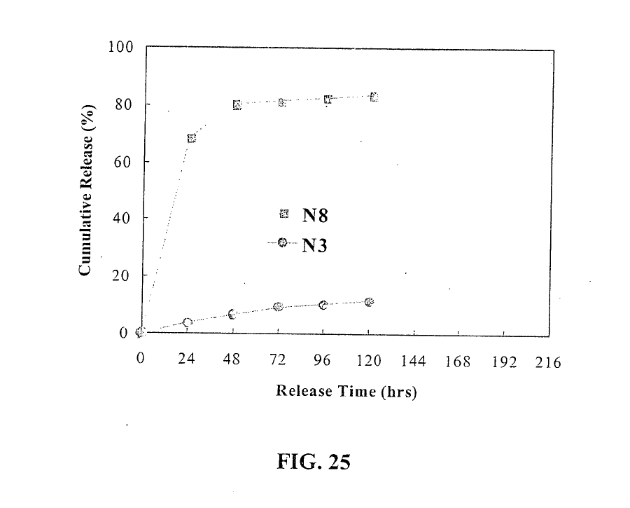

FIG. 25 shows the naltrexone release profiles of compositions having a 20 g/100 g polymer paste drug load and acetic acid as the viscosity reducing agent with a base polymer concentration in the polymer paste of 60% (N8) and 100% (N3).

FIGS. 23, 24 and 25 show that a decrease in the percentage of base polymer present in the sustained release composition results in an increase in both initial release and sustained release.

### EXAMPLE 22

### Mixture of Base Polymers

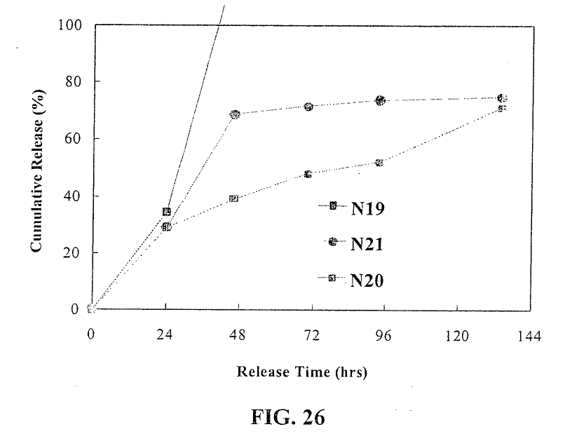

The cumulative release for compositions N19 (60 wt % TEGPLGA+20% DMSO+20% acetic acid), N20 (60 wt % ULMPLGA5050-LA+20% DMSO+20% acetic acid) and N21 (30 wt % ULMPLGA5050LA+30 wt % TEGPLGA+20% DMSO+20% acetic acid) were determined. The cumulative release is depicted graphically in FIG. 26. FIG. 26 shows that the cumulative release of naltrexone employing a combination of base polymers is suitable for use.

### EXAMPLE 23

### Effect of Viscosity Reducing Agents

A: Comparison of PEG200, DMSO and Acetic Acid

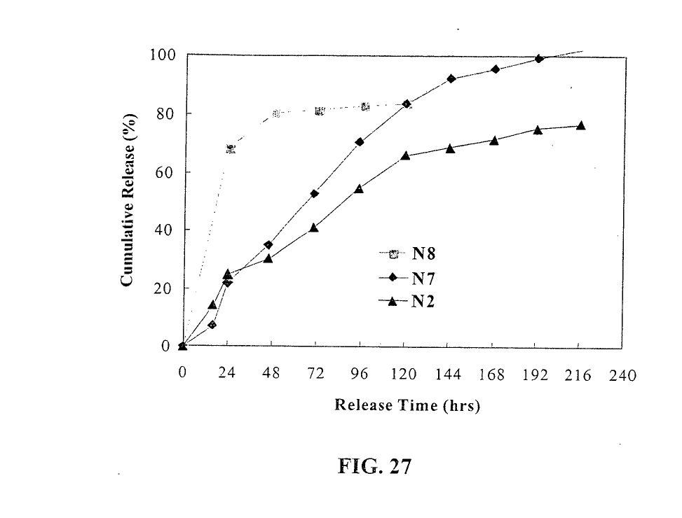

The release of naltrexone from compositions having 40% PEG200, DMSO or acetic acid with 60% ULMPLGA5050-2L (N2, N7 and N8 of Table 9, respectively) was determined and a comparison of the profiles are depicted graphically in FIG. 27. It can be noted from the release profile set forth in FIG. 27 that acetic acid results in a high initial release and that DMSO and PEG200 exhibit similar release profiles.

B: Comparison of Different Combination of Additives

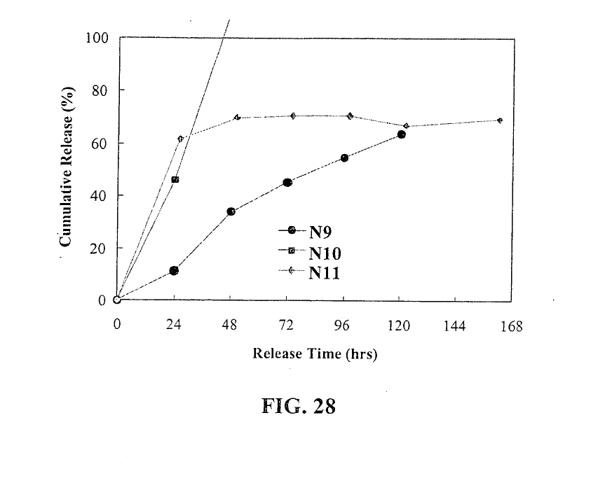

The release of naltrexone from compositions having ULMPLGA5050-2L with different combinations of additives is depicted graphically in FIG. 28. The compositions tested were N9 (20% PEG200+20% DMSO), N10 (20% PEG200+20% acetic acid) and N11 (20% DMSO+20% acetic acid) of Table 9. The graph shows that combinations with PEG200 provides a lower initial release. In addition, it is noted that the use of acetic acid tends to increase initial release.

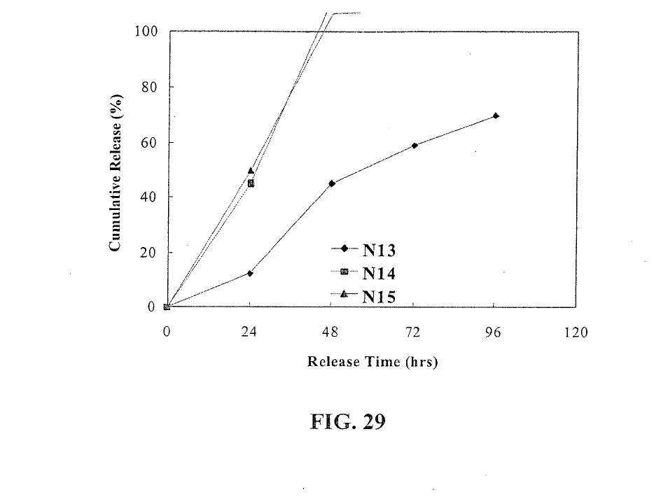

The same comparison as described above and depicted in FIG. 28 was conducted for the polymer ULMPDLLA (compositions N13, N14 and N15 of Table 9). The results are depicted graphically in FIG. 29. Similar to the polymer of FIG. 28, the combination of DMSO and PEG200 provided the lowest initial release of naltrexone from the sustained release composition.

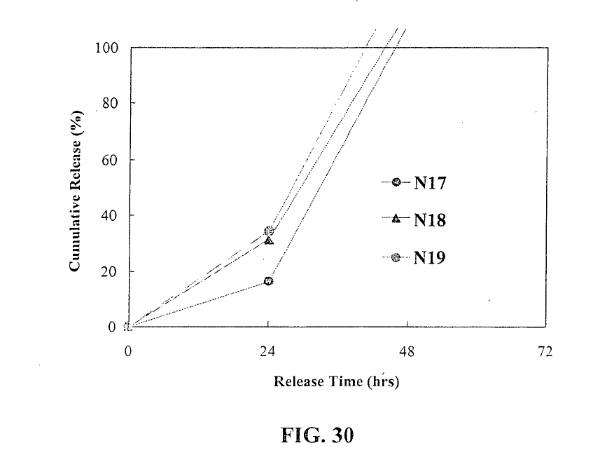

The same comparison as described above and depicted in FIG. 28 was conducted for the polymer TEGPLGA (compositions N17, N18 and N19 of Table 9). The results are depicted graphically in FIG. 30. From FIG. 30 is can be seen the compositions containing TEGPLGA provide a fast sustained release for all viscosity reducing agents tested.

While this invention has been particularly shown and described with references to preferred embodiments thereof, it will be understood by those skilled in the art that various changes in form and details may be made therein without departing from the scope of the invention encompassed by the appended claims.

We claim:

1. A sustained release composition comprising a biologically active agent and a polymer paste comprising a biocompatible, biodegradable polymer having an inherent viscosity of about 0.12 dL/g or less and a viscosity reducing agent, wherein the biocompatible, biodegradable polymer is present in the polymer paste in at least 60% by weight and the viscosity of the composition is about 400 cP or less.

2. The sustained release composition of claim 1, wherein the biocompatible, biodegradable polymer is selected from the group consisting of: poly(lactide)s, poly(glycolide)s, poly(lactide-co-glycolide)s, poly(lactic acid)s, poly(glycolic acid)s, polycarbonates, polyesteramides, polyanhydrides, poly(amino acid)s, polyacetals, polycyanoacrylates, polyetheresters, polyorthoesters, polycaprolactone, poly(dioxanone)s, poly(alkylene alkylate)s, polyurethanes, and blends and copolymers thereof.

3. The sustained release composition of claim 2, wherein the biocompatible, biodegradable polymer is a poly(lactide-co-glycolide) polymer.

4. The sustained release composition of claim 3, wherein the poly(lactide-co-glycolide) polymer is selected from the group consisting of: TEGPLGA5050; ULMPLGA5050-L; ULMPLGA5050-LA; ULMPLGA7525-L; ULMPDLLA-L, and any combination thereof.

5. The sustained release composition of claim 1, wherein the viscosity reducing agent is selected from the group consisting of: polyethylene glycol polymers, surfactants, organic solvents, aqueous solvents, and combinations thereof.

6. The sustained release composition of claim 5, wherein the viscosity reducing agent is a polyethylene glycol polymer.

7. The sustained release composition of claim 6, wherein the polyethylene glycol polymer is PEG200.

8. The sustained release composition of claim 5, wherein the viscosity reducing agent is a polymer surfactant.

9. The sustained release composition of claim 8, wherein the polymer surfactant is a nonionic polymer surfactant selected from the group consisting of poloxamers and polysorbates.

10. The sustained release composition of claim 9, wherein the poloxamer is selected from the group consisting of: poloxamer 407, poloxamer 188, poloxamer 331, poloxamer 184, and combinations thereof.

11. The sustained release composition of claim 1, wherein the viscosity reducing agent is an organic solvent or an aqueous solvent.

12. The sustained release composition of claim 11, wherein the organic solvent is selected from the group consisting of: organic acids; DMSO; dimethylsulfone; tetrahydrofuran; N-methyl-2-pyrrolidone (NMP); 2-pyrrolidone; alcohols; dialkylamides; triacetiens; benzyl benzoate; methyl benzoate; ethyl acetate; ethyl lactate; and combinations thereof.

13. The sustained release composition of claim 12, wherein the dialkylamide is dimethylformamide, dimethylacetamide, or a combination thereof.

14. The sustained release composition of claim 12, wherein the alcohol is solketal, glycerol formal, glycofurol, benzyl alcohol, or a combination thereof.

15. The sustained release composition of claim 12, wherein the organic acid is lactic acid, acetic acid, or a combination thereof.

16. The sustained release composition of claim 5, wherein the viscosity reducing agent is a combination of a polyethylene glycol polymer and an organic solvent.

17. The sustained release composition of claim 5, wherein the viscosity reducing agent is a combination of a polyethylene glycol polymer and an organic acid.

18. The sustained release composition of claim 5, wherein the viscosity reducing agent is a combination of organic solvents.

19. The sustained release composition of claim 1, wherein the biologically active agent is present from about 0.5 g per 100 g polymer paste to about 75 g per 100 g polymer paste.

20. A method for the sustained delivery of a biologically active agent to a patient in need thereof comprising administering a therapeutically effective amount of a sustained release composition comprising a biologically active agent and a polymer paste comprising a biocompatible, biodegradable polymer having an inherent viscosity of about 0.12 dL/g or less and a viscosity reducing agent, wherein the biocompatible, biodegradable polymer is present in the polymer paste in at least 60% by weight and the viscosity of the composition is about 400 cP or less.

21. The method of claim 20, wherein the biocompatible, biodegradable polymer is selected from the group consisting of: poly(lactide)s, poly(glycolide)s, poly(lactide-co-glycolide)s, poly(lactic acid)s, poly(glycolic acid)s, polycarbonates, polyesteramides, polyanhydrides, poly(amino acid)s, polyacetals, polycyanoacrylates, polyetheresters, polyorthoesters, polycaprolactone, poly(dioxanone)s, poly(alkylene alkylate)s, polyurethanes, and blends and copolymers thereof.

22. The method of claim 21, wherein the biocompatible, biodegradable polymer is a poly(lactide-co-glycolide) polymer.

23. The method of claim 22, wherein the poly(lactide-co-glycolide) polymer is selected from the group consisting of: TEGPLGA5050; ULMPLGA5050-L; ULMPLGA5050-LA; ULMPLGA7525-L; ULMPDLLA-L, and combinations thereof.

24. The method of claim 20, wherein the viscosity reducing agent is selected from the group consisting of: polyethylene glycol polymers, surfactants, organic solvents, aqueous solvents, and combinations thereof.

25. The method of claim 24, wherein the viscosity reducing agent is a polyethylene glycol polymer.

26. The method of claim 25, wherein the polyethylene glycol polymer is PEG200.

27. The method of claim 24, wherein the viscosity reducing agent is a polymer surfactant.

28. The method of claim 27, wherein the polymer surfactant is a nonionic polymer surfactant selected from the group consisting of: poloxamers and polysorbates.

29. The method of claim 28 wherein the poloxamer is selected from the group consisting of: poloxamer 407, poloxamer 188, poloxamer 184, poloxamer 331, and combinations thereof.

30. The method of claim 20, wherein the viscosity reducing agent is an organic solvent.

31. The method of claim 30, wherein the organic solvent is selected from the group consisting of: organic acids; DMSO; dimethylsulfone; tetrahydrofuran; N-methyl-2-pyrrolidone (NMP); 2-pyrrolidone; alcohols; dialkylamides; triacetiens; benzyl benzoate; methyl benzoate; ethyl acetate; ethyl lactate; and combinations thereof.

32. The method of claim 31, wherein the dialkylamide is dimethylformamide, dimethylacetamide, or a combination thereof.

33. The method of claim 31, wherein the alcohol is solketal, glycerol formal, glycofurol, benzyl alcohol, or a combination thereof.

34. The method of claim 31, wherein the organic acid lactic acid, acetic acid, or a combination thereof.

35. The method of claim 24, wherein the viscosity reducing agent is a combination of a polyethylene glycol polymer and an organic solvent.

36. The method of claim 24, wherein the viscosity reducing agent is a combination of a polyethylene glycol polymer and an organic acid.

37. The method of claim 24, wherein the viscosity reducing agent is a combination of an organic solvent and an organic acid.

38. The method of claim 20 wherein the biologically active agent is present from about 0.5 g per 100 g polymer paste to about 75 g per 100 g polymer paste.

39. A polymer paste comprising a biocompatible, biodegradable polymer having an inherent viscosity of about 0.12 dL/g or less and a viscosity reducing agent, wherein the biocompatible, biodegradable polymer is present in the polymer paste in at least 60% by weight and the viscosity of the paste is about 400 cP or less.

40. The polymer paste of claim 39, wherein the biocompatible, biodegradable polymer is selected from the group consisting of: poly(lactide)s, poly(glycolide)s, poly(lactide-co-glycolide)s, poly(lactic acid)s, poly(glycolic acid)s, polycarbonates, polyesteramides, polyanhydrides, poly(amino acid)s, polyacetals, polycyanoacrylates, polyetheresters, polyorthoesters, polycaprolactone, poly(dioxanone)s, poly(alkylene alkylate)s, polyurethanes, and blends and copolymers thereof.

41. The polymer paste of claim 40, wherein the biocompatible, biodegradable polymer is a poly(lactide-co-glycolide) polymer.

42. The polymer paste of claim 41, wherein the poly(lactide-co-glycolide) polymer is selected from the group consisting of: TEGPLGA5050; ULMPLGA5050-L; ULMPLGA5050-LA; ULMPLGA7525-L; ULMPDLLA-L, and any combination thereof.

43. The polymer paste of claim 39, wherein the viscosity reducing agent is selected from the group consisting of: polyethylene glycol polymers, surfactants, organic solvents, aqueous solvents, and combinations thereof.

44. The polymer paste of claim 43, wherein the viscosity reducing agent is a polyethylene glycol polymer.

45. The polymer paste of claim 44, wherein the polyethylene glycol polymer is PEG200.

46. The polymer paste of claim 43, wherein the viscosity reducing agent is a polymer surfactant.

47. The polymer paste of claim 46, wherein the polymer surfactant is a nonionic polymer surfactant selected from the group consisting of: poloxamers and polysorbates.

48. The polymer paste of claim 47, wherein the poloxamer is selected from the group consisting of: poloxamer 407, poloxamer 188, poloxamer 331, poloxamer 184, and combinations thereof.

49. The polymer paste of claim 39, wherein the viscosity reducing agent is an organic solvent or an aqueous solvent.

50. The polymer paste of claim 49, wherein the organic solvent is selected from the group consisting of: organic acids; DMSO; dimethylsulfone; tetrahydrofuran; N-methyl-2-pyrrolidone (NMP); 2-pyrrolidone; alcohols; dialkylamides; triacetiens; benzyl benzoate; methyl benzoate; ethyl acetate; ethyl lactate; and combinations thereof

51. The polymer paste of claim 50, wherein the dialkylamide is dimethylformamide, dimethylacetamide, or a combination thereof.

52. The polymer paste of claim 50, wherein the alcohol is solketal, glycerol formal, glycofurol, benzyl alcohol, or a combination thereof.

53. The polymer paste of claim 50, wherein the organic acid is lactic acid, acetic acid, or a combination thereof.

54. The polymer paste of claim 43 wherein the viscosity reducing agent is a combination of a polyethylene glycol polymer and an organic solvent.

55. The polymer paste of claim 43, wherein the viscosity reducing agent is a combination of a polyethylene glycol polymer and an organic acid.

56. The polymer paste of claim 43, wherein the viscosity reducing agent is a combination of organic solvents.
